(19) United States
(12) Patent Application Publication
(10) Pub. No.: US 2005/0136536 A1
(43) Pub. Date: Jun. 23, 2005

(54) Embryonic epithelial cells

(76) Inventors: Daniel G. Anderson, Framingham, MA (US); Shulamit Levenberg, Haifa (IL); Robert S. Langer, Newton, MA (US)

Correspondence Address: CHOATE, HALL & STEWART LLP, EXCHANGE PLACE, 53 STATE STREET, BOSTON, MA, 02109, US

(21) Appl. No.: 10/941,390

(22) Filed: Sep. 15, 2004

Related U.S. Application Data
(60) Provisional application No. 60/570,187, filed on May 12, 2004.
(60) Provisional application No. 60/503,165, filed on Sep. 15, 2003.

(51) Int. Cl. C12N 5/08

(52) U.S. Cl. 435/366

(57) ABSTRACT

A population of embryonic epithelial cells produced in vitro from embryonic stem cells. In one embodiment, at least 45% of the cells express cytokeratin, for example, cytokeratin-7.

This application claims the priority of Provisional application No. 60/570,187, filed May 12, 2004, and provisional application No. 60/503,165, filed Sep. 15, 2003, the entire contents of both of which are incorporated by reference herein.

### FIELD OF THE INVENTION

This invention pertains to the use of embryonic stem cells, and, more specifically, to the differentiation, isolation, and characterization of human embryonic epithelial cells.

### BACKGROUND OF THE INVENTION

The surface on which cells grow and the extracellular microenvironment play a key role in controlling cellular behavior (A. Spradling, et al., Nature 414, 98-104 (2001); C. Streuli, Curr Opin Cell Biol 11, 634-640 (1999)). Properties such as surface roughness, hydrophobicity, and specific interaction with the cell surface, can all affect cell behavior (W. M. Saltzman, et al., “Principles of tissue engineering”, Academic Press 221-235 (2000)). The effects of the cellular substrate are also important factors in biomaterial-based therapies. Tissue engineered constructs, ex-vivo cell isolation, bio-reactors and cell encapsulation require some type of interaction between cells and supporting material for growth, function, and/or delivery (R. P. Lanzo, et al., “Principles of tissue engineering”, Academic Press, ed. 2^{nd} (2000)). Much research is currently focused on the development of biomaterials that provide optimal cellular substrates, including the development of bioactive materials through the incorporation of ligands, and encapsulation of DNA and growth factors (R. R. Chen, et al., Pharmaceutical Research 20, 1103-1112 (2003); S. E. Sakiyama-Elbert, et al., Annual Review of Materials Research 31, 183-201 (2001)).

The application of stem cells, including human embryonic stem cells (hES cells), in tissue engineering and cell therapy requires the ability to control the growth and differentiation of these cells into useful cell types. However, the effects of biomaterials on stem cell behavior has not been studied in great detail, in part due to the large potential polymeric diversity and the lack of systems allowing for easy synthesis and testing of material-cell interactions. To address this need, we sought to develop a miniaturized system for the synthesis and screening of cell-polymer interactions.

### Definitions

The term embryonic epithelial cell refers to a partially differentiated cell that may differentiate to an epithelial cell under appropriate in vivo or in vitro conditions. Embryonic epithelial cells may be identified by expression of genes or production of proteins characteristic of epithelial cells, for example, cytokeratin. Cytokeratins are a family of proteins that are found in epithelial tissue in various parts of the body. Different tissues may include one or more of over two dozen cytokeratins. For example, cytokeratin 7 is found in lung and breast epithelium but not colon and prostate epithelium. Cytokeratin 20 is found in gastric and intestinal epithelium.

The term alkyl as used herein refers to saturated, straight- or branched-chain hydrocarbon radicals derived from a hydrocarbon moiety containing between one and twenty carbon atoms by removal of a single hydrogen atom. Examples of alkyl radicals include, but are not limited to, methyl, ethyl, propyl, isopropyl, n-butyl, tert-butyl, n-pentyl, neopentyl, n-hexyl, n-heptyl, n-octyl, n-decyl, n-undecyl, and dodecyl.

The term alkoxy as used herein refers to an alkyl groups, as previously defined, attached to the parent molecular moiety through an oxygen atom. Examples include, but are not limited to, methoxy, ethoxy, propoxy, isopropoxy, n-butoxy, tert-butoxy, neopentoxy, and n-hexoxy.

The term alkenyl denotes a monovalent group derived from a hydrocarbon moiety having at least one carbon-carbon double bond by the removal of a single hydrogen atom. Alkenyl groups include, for example, ethenyl, propenyl, butenyl, 1-methyl-2-buten-1-yl, and the like.

The term alkynyl as used herein refers to a monovalent group derived form a hydrocarbon having at least one carbon-carbon triple bond by the removal of a single hydrogen atom. Representative alkynyl groups include ethynyl, 2-propynyl (propargyl), 1-propynyl, and the like.

The term alkylamino, dialkylamino, and trialkylamino as used herein refers to one, two, or three, respectively, alkyl groups, as previously defined, attached to the parent molecular moiety through a nitrogen atom. The term alkylamino refers to a group having the structure —NHR′ wherein R′ is an alkyl group, as previously defined; and the term dialkylamino refers to a group having the structure —NR′R″, wherein R′ and R″ are each independently selected from the group consisting of alkyl groups. The term trialkylamino refers to a group having the structure —NR′R″R′″, wherein R′, R″, and R′″ are each independently selected from the group consisting of alkyl groups. Additionally, R′, R″, and/or R′″ taken together may optionally be —(CH₂)ₖ— where k is an integer from 2 to 6. Example include, but are not limited to, methylamino, dimethylamino, ethylamino, diethylamino, diethylaminocarbonyl, methylethylamino, iso-propylamino, piperidino, trimethylamino, and propylamino.

The terms alkylthioether and thioalkoxyl refer to an alkyl group, as previously defined, attached to the parent molecular moiety through a sulfur atom.

The term aryl as used herein refers to carbocyclic ring system having at least one aromatic ring including, but not limited to, phenyl, naphthyl, tetrahydronaphthyl, indanyl, indenyl, and the like. Aryl groups can be unsubstituted or substituted with substituents selected from the group consisting of branched and unbranched alkyl, alkenyl, alkynyl, haloalkyl, alkoxy, thioalkoxy, amino, alkylamino, dialkylamino, trialkylamino, acylamino, cyano, hydroxy, halo, mercapto, nitro, carboxyaldehyde, carboxy, alkoxycarbonyl, and carboxamide. In addition, substituted aryl groups include tetrafluorophenyl and pentafluorophenyl.

The term carboxylic acid as used herein refers to a group of formula —CO₂H.

The terms halo and halogen as used herein refer to an atom selected from fluorine, chlorine, bromine, and iodine.

The term heterocyclic, as used herein, refers to a non-aromatic partially unsaturated or fully saturated 3- to 10-membered ring system, which includes single rings of 3 to 8 atoms in size and bi- and tri-cyclic ring systems which may include aromatic six-membered aryl or aromatic heterocyclic groups fused to a non-aromatic ring. These heterocyclic rings include those having from one to three heteroatoms independently selected from oxygen, sulfur, and nitrogen, in which the nitrogen and sulfur heteroatoms may optionally be oxidized and the nitrogen heteroatom may optionally be quaternized.

The term aromatic heterocyclic, as used herein, refers to a cyclic aromatic radical having from five to ten ring atoms of which one ring atom is selected from sulfur, oxygen, and nitrogen; zero, one, or two ring atoms are additional heteroatoms independently selected from sulfur, oxygen, and nitrogen; and the remaining ring atoms are carbon, the radical being joined to the rest of the molecule via any of the ring atoms, such as, for example, pyridyl, pyrazinyl, pyrimidinyl, pyrrolyl, pyrazolyl, imidazolyl, thiazolyl, oxazolyl, isooxazolyl, thiadiazolyl, oxadiazolyl, thiophenyl, furanyl, quinolinyl, isoquinolinyl, and the like.

Specific heterocyclic and aromatic heterocyclic groups that may be included in the compounds of the invention include: 3-methyl-4-(3-methylphenyl)piperazine, 3 methylpiperidine, 4-(bis-(4-fluorophenyl)methyl)piperazine, 4-(diphenylmethyl)piperazine, 4-(ethoxycarbonyl)piperazine, 4-(ethoxycarbonylmethyl)piperazine, 4-(phenylmethyl)piperazine, 4-(1-phenylethyl)piperazine, 4-(1,1-dimethylethoxycarbonyl)piperazine, 4-(2-(bis-(2-propenyl)amino)ethyl)piperazine, 4-(2-(diethylamino)ethyl)piperazine, 4-(2-chlorophenyl)piperazine, 4-(2-cyanophenyl)piperazine, 4-(2-ethoxyphenyl)piperazine, 4-(2-ethylphenyl)piperazine, 4-(2-fluorophenyl)piperazine, 4-(2-hydroxyethyl)piperazine, 4-(2-methoxyethyl)piperazine, 4-(2-methoxyphenyl)piperazine, 4-(2-methylphenyl)piperazine, 4-(2-methylthiophenyl)piperazine, 4-(2-nitrophenyl)piperazine, 4-(2-nitrophenyl)piperazine, 4-(2-phenylethyl)piperazine, 4-(2-pyridyl)piperazine, 4-(2-pyrimidinyl)piperazine, 4-(2,3-dimethylphenyl)piperazine, 4-(2,4-difluorophenyl)piperazine, 4-(2,4-dimethoxyphenyl)piperazine, 4-(2,4-dimethylphenyl)piperazine, 4-(2,5-dimethylphenyl)piperazine, 4-(2,6-dimethylphenyl)piperazine, 4-(3-chlorophenyl)piperazine, 4-(3-methylphenyl)piperazine, 4-(3-trifluoromethylphenyl)piperazine, 4-(3,4-dichlorophenyl)piperazine, 4-3,4-dimethoxyphenyl)piperazine, 4-(3,4-dimethylphenyl)piperazine, 4-(3,4-methylenedioxyphenyl)piperazine, 4-(3,4,5-trimethoxyphenyl)piperazine, 4-(3,5-dichlorophenyl)piperazine, 4-(3,5-dimethoxyphenyl)piperazine, 4-(4-(phenylmethoxy)phenyl)piperazine, 4-(4-(3,1-dimethylethyl)phenylmethyl)piperazine, 4-(4-chloro-3-trifluoromethylphenyl)piperazine, 4-(4-chlorophenyl)-3-methylpiperazine, 4-(4-chlorophenyl)piperazine, 4-(4-chlorophenyl)piperazine, 4-(4-chlorophenylmethyl)piperazine, 4-(4-fluorophenyl)piperazine, 4-(4-methoxyphenyl)piperazine, 4-(4-methylphenyl)piperazine, 4-(4-nitrophenyl)piperazine, 4-(4-trifluoromethylphenyl)piperazine, 4-cyclohexylpiperazine, 4-ethylpiperazine, 4-hydroxy-4-(4-chlorophenyl)methylpiperidine, 4-hydroxy-4-phenylpiperidine, 4-hydroxypyrrolidine, 4-methylpiperazine, 4-phenylpiperazine, 4-piperidinylpiperazine, 4-(2-furanyl)carbonyl)piperazine, 4-((1,3-dioxolan-5-yl)methyl)piperazine, 6-fluoro-1,2,3,4-tetrahydro-2-methylquinoline, 1,4-diazacylcloheptane, 2,3-dihydroindolyl, 3,3-dimethylpiperidine, 4,4-ethylenedioxypiperidine, 1,2,3,4-tetrahydroisoquinoline, 1,2,3,4-tetrahydroquinoline, azacyclooctane, decahydroquinoline, piperazine, piperidine, pyrrolidine, thiomorpholine, and triazole.

The term carbamoyl, as used herein, refers to an amide group of the formula —CONH₂.

The term hydrocarbon, as used herein, refers to any chemical group comprising hydrogen and carbon. The hydrocarbon may be substituted or unsubstitued. The hydrocarbon may be unsaturated, saturated, branched, unbranched, cyclic, polycyclic, or heterocyclic. Illustrative hydrocarbons include, for example, methyl, ethyl, n-propyl, iso-propyl, cyclopropyl, allyl, vinyl, n-butyl, tert-butyl, ethynyl, cyclohexyl, methoxy, diethylamino, and the like. As would be known to one skilled in this art, all valencies must be satisfied in making any substitutions.

The terms substituted, whether preceded by the term “optionally” or not, and substituent, as used herein, refer to the ability, as appreciated by one skilled in this art, to change one functional group for another functional group provided that the valency of all atoms is maintained. When more than one position in any given structure may be substituted with more than one substituent selected from a specified group, the substituent may be either the same or different at every position. The substituents may also be further substituted (e.g., an aryl group substituent may have another substituent off it, such as another aryl group, which is further substituted with fluorine at one or more positions).

The term ureido, as used herein, refers to a urea groups of the formula —NH—CO—NH₂.

### SUMMARY OF THE INVENTION

In one aspect, the invention is a population of embryonic epithelial cells produced in vitro from embryonic stem cells. At least 45%, 55%, 65%, 75%, 85%, or 95% of the cells may express cytokeratin, for example, cytokeratin 7.

In another aspect, the invention is a population of cytokeratin 7-positive cell produced in vitro from embryonic stem cells.

In another aspect, the invention is a population of cytokeratin or cytokeratin 7-positive cells produced by the step of exposing a population of embryonic stem cells to retinoic acid. The population may be exposed to retinoic acid in the presence of serum, and the population of embryonic stem cells may be seeded on a cell support substrate.

In another aspect, the invention is a composition comprising a cell support substrate and human embryonic epithelial cells supported by the cell support substrate. The composition may further include retinoic acid or serum.

In another aspect, the invention is a method of enriching a population of embryonic stem cells with epithelial-like cells. The method includes providing a population of human embryonic stem cells and culturing the stem cells on an acrylate polymer in a culture-medium including retinoic acid. The culture medium may include serum. Providing a population of human embryonic stem cells may include culturing embryonic stem cells under conditions where embryoid bodies are formed and dissociating the embryoid bodies.

### BRIEF DESCRIPTION OF THE DRAWING

The invention is described with reference to the several figures of the drawing, in which,

FIG. 1A is a schematic of an exemplary polymer microarray produced using the techniques of the invention;

FIG. 1B is a schematic of an alternative polymer microarray produced using the techniques of the invention;

FIG. 2A depicts monomers employed to make microarrays according to an embodiment of the invention;

FIG. 2B is a diagram indicating the distribution of monomers in the array to form copolymers;

FIG. 2C is an image of a polymer array in triplicate provided by an Arrayworx reader (red box: 70% 1; yellow box: 70% 6);

FIG. 2D is a DIC light micrograph of a typical polymer element overlayed with a few fluorescent cells (red);

FIG. 3 is a schematic view of an exemplary apparatus for use with the invention;

FIG. 4A is an image of a polymer array in triplicate incubated with hES EB day 6 cells in the presence of retinoic acid for 6 days and then stained for cytokeratin 7 (green) and vimentin (red) (polymer elements are blue);

FIG. 4B is a larger scale view of one of the arrays depicted in FIG. 4A;

FIG. 4C is a yet higher scale view of the array depicted in FIGS. 4A and 4B;

FIG. 4D illustrates cell nuclei in the array of FIGS. 4A-C revealed by green fluorescence;

FIG. 4E is an image of a cytokeratin 7-positive spot on a polymer produced from monomer 9;

FIG. 4F is a graph showing cell growth as a function of polymer composition, measured as the average percent coverage of a polymer spot by cells;

FIG. 5A is a diagram indicating the composition of polymers in the array shown in FIG. 5B;

FIG. 5B is an image of a polymer array produced according to the diagram in FIG. 5A.

FIGS. 6A, C-E are images of hES cells grown on a polymer array in the absence of retinoic acid for 6 days and then stained for cytokeratin 7 (green) and vimentin (red) (polymer spots and unstained cells are blue);

FIGS. 6B, F-H are images of hES cells grown on a polymer array in the presence of retinoic acid for 6 days and then stained for cytokeratin 7 (green) and vimentin (red);

FIGS. 6I-K are an image of hES cells grown on a polymer array in the absence of retinoic acid for 24 hours and then stained for cytokeratin 7 (green) and vimentin (red);

FIGS. 6L-N are images of hES cells grown on a polymer array in the presence of retinoic acid for 24 hours and then stained for cytokeratin 7 (green) and vimentin (red);

FIG. 7 provides images and data for hES cells grown on “hit” polymer arrays (see FIG. 5A) for 1 or 6 days and stained for cytokeratin 7 (green), vimentin (red), and DNA (blue) (cells per spot and percent cells site of keratin positive calculated after 6 days exposure to retinoic acid);

FIG. 8A is an image of C2C12 cells seated onto a polymer array and stained after 6 days for actin (red), myogenin (green), and DNA (blue);

FIG. 8B is a larger scale view of one of the arrays illustrated in triplicate in FIG. 8A;

FIGS. 8C-E are images of cells on polymers produced from 70% 14 and from left to right, 30% 1, 30% 2, 30% 3, 30% 25, 30% 8, and 30% 9;

FIG. 8F is an image of cells grown on a polymer produced from 70% 14 and 30% 8;

FIG. 8G is a high magnification fluorescence image of a typical polymer element;

FIG. 9 is a series of fluorescence images of cells cultured on gelatin in a growth medium including A serum, cells stained after 6 days; B serum plus retinoic acid, cells stained after 6 days; C serum plus retinoic acid, cells stained after 6 days; D serum plus AM580, cells stained after 6 days; E serum, cells stained after 8 days; F serum plus retinoic acid, cells stained after 8 days; G serum plus AM580, cells stained after 8 days.

### DETAILED DESCRIPTION OF CERTAIN PREFERRED EMBODIMENTS

In one embodiment, the invention provides a method of enriching a population of embryonic stem cells with epithelial-like cells. The method includes providing a population of human embryonic stem cells and culturing the stem cells on an acrylate polymer in a culture medium including retinoic acid. Microarrays of polymers may be employed to select polymers which facilitate proliferation and differentiation of the cells.

Polymer Microarrays

The present invention exploits polymer microarrays such as those disclosed in U.S. patent applications Ser. Nos. 10/214,723 and 09/803,319, published as 2004-0028804 and 2002-0142304, respectively. The techniques of the invention may be exploited to produce a cell-compatible, miniaturized polymer array characterized by the ability to synthesize a large number of materials in nanoliter volumes, polymer elements that are attached to the microarray in a manner that would be compatible with those materials and resistant to the aqueous conditions necessary for cell-based testing, inhibition of cell growth in the spaces between different polymers to allow material effects on cells to be independent of neighboring materials, and a format that allows simple, simultaneous assay of multiple cellular markers.

In one embodiment, a substrate surface is treated to render it cytophobic, for example, by coating it first with epoxide and then with poly(hydroxyethyl methacrylate) (pHEMA). pHEMA inhibits cell growth (J. Folkman, et al., Nature 273, 345-349 (1978)), and a monomer deposited on a pHEMA surface may interpenetrate and potentially become fixed in place upon polymerization. Other polymers that may be used to form cytophobic surfaces include poly alkylene glycols such as poly(ethylene glycol) and its co-polymers. Alternatively, polymers derivatized with poly(ethylene glycol) or other poly(alkylene glycols) may be employed.

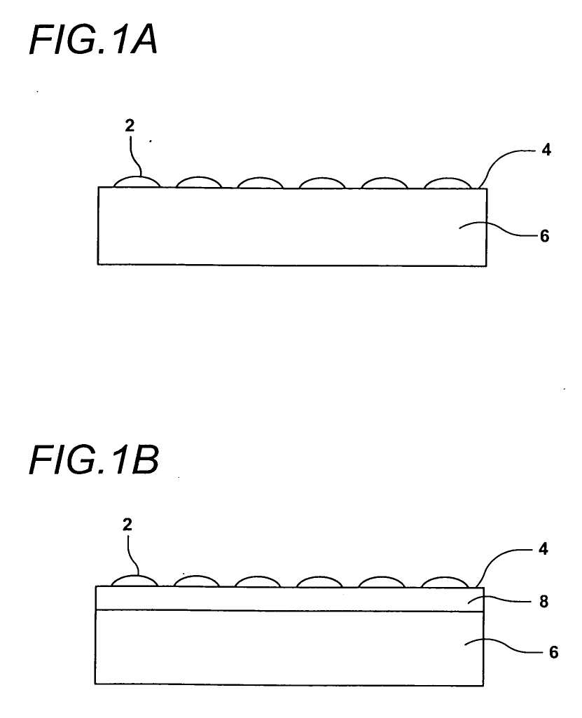

Polymer elements are produced on the surface by depositing an array of monomers and then polymerizing them in situ. The polymer elements may be associated with the substrate surface via non-covalent interactions such as chemical adsorption, hydrogen bonding, surface interpenetration, ionic bonding, van der Waals forces, hydrophobic interactions, dipole-dipole interactions, mechanical interlocking, and combinations of these; however, the polymer elements may also be associated with the substrate surface via covalent interactions. The base can be a glass, plastic, metal, or ceramic, but can also be made of any other suitable material. FIG. 1A shows an embodiment of an array of polymer elements 2 disposed on a surface 4 of substrate 6. FIG. 1B illustrates an embodiment in which a coating 8 is disposed on substrate 6, and polymer elements 2 are disposed on surface 4, which is the surface of the coating.

The substrate surface material should be chosen to maximize adherence of the polymer elements while controlling spreading of the deposited monomer. Where cell-polymer interactions are studied, a cytophobic coating will prevent migration of cells from one polymer element to another. An epoxy coating interposed between the cytophobic coating and the base may increase the adherence of the coating to the base. The synthesis of polymers in arrayed form onto a conventional 25×75 mm glass slide allows for easy, simultaneous staining and four-color fluorescence imaging of multiple slides.

Once the substrate surface has been provided, monomers are deposited on the surface and polymerized to form a microarray of polymer elements. In one embodiment, liquid monomers diluted in 25% dimethylformamide (DMF) are deposited on the substrate. The solvent decreases the viscosity of the monomers and facilitates deposition of a precise amount of monomer. The amount of solvent or the solvent itself may be changed to alter the viscosity as needed. Alternative solvents include but are not limited to dimethylsulfoxide, chloroform, dichlorobenzene, and other chlorinated solvents.

In one embodiment, the monomer is part of a biocompatible polymer. A number of biodegradable and non-biodegradable biocompatible polymers are known in the field of polymeric biomaterials, controlled drug release and tissue engineering (see, for example, U.S. Pat. Nos. 6,123,727; 5,804,178; 5,770,417; 5,736,372; 5,716,404 to Vacanti; U.S. Pat. Nos. 6,095,148; 5,837,752 to Shastri; U.S. Pat. No. 5,902,599 to Anseth; U.S. Pat. Nos. 5,696,175; 5,514,378; 5,512,600 to Mikos; U.S. Pat. No. 5,399,665 to Barrera; U.S. Pat. No. 5,019,379 to Domb; U.S. Pat. No. 5,010,167 to Ron; U.S. Pat. No. 4,946,929 to d'Amore; and U.S. Pat. Nos. 4,806,621; 4,638,045 to Kohn; see also Langer, Acc. Chem. Res. 33: 94, 2000; Langer, J. Control Release 62: 7, 1999; and Uhrich et al., Chem. Rev. 99: 3181, 1999; all of which are incorporated herein by reference). Exemplary biocompatible polymer classes that may be incorporated into polymer elements 2 using the techniques of the invention include polyamides, polyphosphazenes, polypropylfumarates, synthetic poly(amino acids), polyethers, polyacetals, polycyanoacrylates, polyurethanes, polycarbonates, polyanhydrides, poly(ortho esters), polyhydroxyacids, polyesters, polyacrylates, ethylene-vinyl acetate polymers, cellulose acetates, polystyrenes, poly(vinyl chloride), poly(vinyl fluoride), poly(vinyl imidazole), poly(vinyl alcohol), and chlorosulphonated polyolefins. The term biodegradable, as used herein, refers to materials that are enzymatically or chemically (e.g., hydrolytically) degraded in vivo into simpler chemical species. Monomers that are used to produce these polymers are easily purchased from companies such as Polysciences, Sigma, Scientific Polymer Products, and Monomer-Polymer & Dajac Laboratories. These monomers may be combined in an array to form a wide variety of co-polymers.

The monomers may polymerize by chain polymerization. Exemplary monomers subject to radical chain polymerization include ethylene, vinyl derivatives of ethylene, including but not limited to vinyl acetate, vinyl chloride, vinyl alcohol, and vinyl benzene (styrene), vinylidine derivatives of ethylene, including but not limited to vinylidine chloride, acrylates, methacrylates, acrylonitriles, acrylamides, acrylic acid, and methacrylic acid, fluoropolymers, dienes, including but not limited to butadiene, isoprene, and their derivatives, and aromatic monomers such as phenylene and its derivatives, such as phenylene vinylene. Monomers such as α-olefins, 1,1-dialkyl olefins, vinyl ethers, aldehydes, and ketones may be polymerized by anionic chain polymerization, cationic chain polymerization, or both. Additional monomers can be found in George Odian's Principles of Polymerization, (3rd Edition, 1991, New York, John Wiley and Sons), the entire contents of which are incorporated herein by reference.

One skilled in the art will recognize that the techniques of the invention may also be exploited to produce microarrays by step polymerization. The reaction conditions for a variety of polyesters, polyamides, polyurethanes, and other condensation polymers are well known in the art (see Odian, 1991). Such reactions may be easily adapted to produce microarrays on substrates. In one embodiment, neat monomers are deposited as a liquid or in a solution with a solvent such as DMSO or chloroform to prevent premature precipitation of the polymer. Non-volatile solvents are preferred to reduce evaporation. Alternatively or in addition, a catalyst, for example, sulfuric acid or p-toluenesulfonic acid, may be used to increase the rate of reaction. The substrate may be heated or placed in a low pressure atmosphere to drive off the condensation product and drive the reaction. The low volume and high surface area of the droplets should facilitate the removal of the condensation product without the use of purging gases or high vacuum conditions.

Monomers that require chemical initiators may also be used. If the initiator works at a specific temperature, the monomer solutions should be cooled during deposition and then warmed to initiate polymerization. It may be desirable to use a less viscous solvent than would be employed to deposit the microarray at room temperature. In an alternative embodiment, monomers may be deposited in a microarray and then exposed to an ozone atmosphere to initiate polymerization.

The molecular weight of the resultant polymer may be controlled by adjusting the properties of the solvent. Modifying the viscosity of the solvent changes the polymerization rate and the resulting molecular weight distribution. Some solvents provide a more favorable environment for radicals and intermediate products formed during polymerization and allow polymerization to continue for a longer time before termination. The selection of solvents to stabilize or destabilize radicals or to promote condensation and other step polymerization reactions is well known to those skilled in the art.

In an alternative embodiment, the molecular weight of the polymer may be controlled by varying the concentration of monomer in the stock solution or the ratios of difunctional monomers to unifunctional monomers. Increased concentrations of difunctional monomers will increase the degree of cross-linking in the chains. Monofunctional monomers may be modified to form difunctional monomers by reacting them with a linker chain. Appropriate linkers and chemical reactions will be evident to one skilled in the art. For example, dicarboxylic acids are reactive with a wide variety of functional groups commonly incorporated into vinyl monomers, including alcohols, amines, and amides.

In one embodiment, acrylate monomers are used to produce the polymer arrays of the invention. A variety of acrylate-based polymers have been used for tissue engineering, surgical glues, and drug delivery (J. P. Fisher, et al., Annu. Rev. Mater. Res. 31, 171-181 (2001)). There are a number of commercially available acrylate monomers, and these can be polymerized quickly using a light-activated radical initiator. In one embodiment, acrylate monomers having the structure 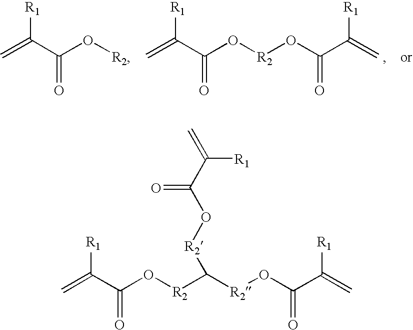
are used to produce polymer elements for use with the invention. R₁ may be methyl or hydrogen. R₂, R₂′, and R₂″ may include alkyl, aryl, heterocycles, cycloalkyl, aromatic heterocycles, multicycloalkyl, hydroxyl, ester, ether, halide, carboxylic acid, amino, alkylamino, dialkylamino, trialkylamino, amido, carbamoyl thioether, thiol, alkoxy, or ureido groups. R₂, R₂′, and R₂″ may also include branches or substituents including alkyl, aryl, heterocycles, cycloalkyl, aromatic heterocycles, multicycloalkyl, hydroxyl, ester, ether, halide, carboxylic acid, amino, alkylamino, dialkylamino, trialkylamino, amido, carbamoyl, thioether, thiol, alkoxy, or ureido groups. In one embodiment, monomers are sufficiently stable that they can be deposited on the slide and sit for a moment, e.g., 30 seconds to 1 or 2 minutes, before being polymerized after exposure to UV light.

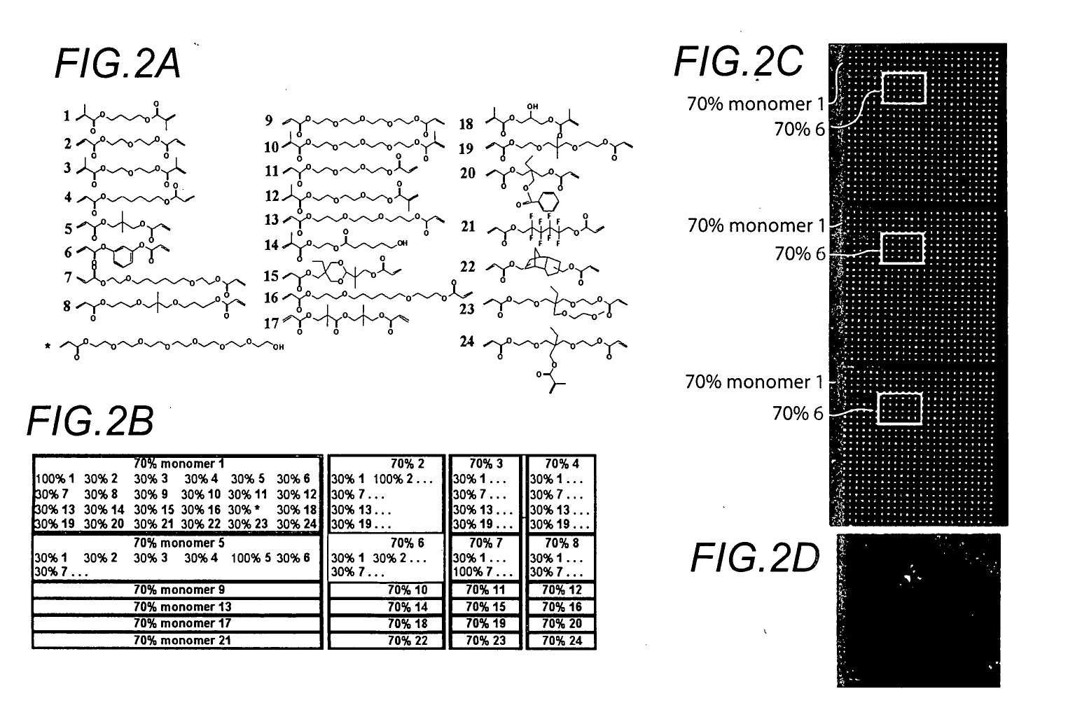

Exemplary acrylate monomers, including bifunctional and multifunctional acrylates for use with the invention are listed in Table 1 and shown in FIG. 2A. These may be purchased from Sigma-Aldrich (Milwaukee, Wis.), Scientific Polymer Products (Onterio, N.Y.), and Polysciences (Warrington, Pa.). In one embodiment, these monomers are diluted by 25% with DMF before spotting to reduce their viscosity and ensure reproducible deposition onto the substrate (see Examples). One skilled in the art will recognize that mixtures of multifunctional and monofunctional monomers may be used to control the degree of cross-linking in the polymer.

TABLE 1

| | Pictured in |
|---|---|
| Diacrylate species | |
| 1,4 butanediol dimethacrylate | 1 |
| diethylene glycol diacrylate | 2 |
| diethylene glycol dimethacrylate | 3 |
| 1,6 hexanediol diacrylate | 4 |
| neopentyl glycol diacrylate | 5 |
| phenylene diacrylate 1,3 | 6 |
| propoxylated neopentyl glycol diacrylate | 8 |
| tetraethylene glycol diacrylate | 9 |
| tetraethylene glycol dimethacrylate | 10 |
| triethylene glycol diacrylate | 11 |
| triethylene glycol dimethacrylate | 12 |
| tripropylene glycol diacrylate | 13 |
| caprolactone 2-(methacryloyloxy)ethyl ester | 14 |
| 5-ethyl-5-(hydroxymethyl)-β,β-dimethyl-1,3-dioxane- | 15 |
| 2-ethanol diacrylate | |
| 1,6-hexanediol propoxylate diacrylate | 16 |
| 3-hydroxy-2,2-dimethylpropyl 3-hydroxy-2,2- | |
| dimethylpropionate diacrylate | |
| glycerol 1,3-diglycerolate diacrylate | |
| glycerol dimethacrylate, mixture of isomers, tech. | |
| 85%, neopentyl glycol dimethacrylate | |
| neopentyl glycol ethoxylate (1 EO/OH) diacrylate | 19 |
| trimethylolpropane benzoate diacrylate | 20 |
| 1,14-tetradecanediol dimethacrylate | |
| tricyclo[5.2.1.0^{2,6}]decanedimethanol diacrylate | 22 |
| trimethylolpropane ethoxylate (1 EO/OH) methyl ether | |
| diacrylate | |
| trimethylolpropane triacrylate, tech. | |

Using the monomers described above, one skilled in the art may adjust many properties of the resulting polymer. For example, both ester and ether groups contributed to the hydrophilicity of the resulting polymer, but they contribute different amounts of electron density. Likewise, the use of amino and thio groups varies the electron density of the resulting polymer differently than oxygenated functional groups. By varying the number of ether groups in the monomer and the length of the R₂ (including R₂′ and R₂″) group, e.g., the distance between the ester linkages, the skilled artisan may tailor the electron density of the polymer. Branched monomers also change electron density by allowing more ether groups to fit in an R₂ group of a certain length, by changing the packing density of the resulting polymer, or both. The use of cyclic moieties and aromatic moieties also changes the electron density of R₂. An R₁ methyl group contributes more electron density to the ester group that a hydrogen atom. In addition, the cross-link density of the polymer may be adjusted by varying the proportion of monofunctional, bifunctional, and other multifunctional monomers. The use of a co-monomer enables fine tuning of the electron density of the polymer. Both the composition and the amount of the co-monomer may be varied to adjust the hydrophobicity or hydrophilicity of the resulting polymer.

Once the appropriate monomer and the substrate surface have been selected for use in the present invention, it will be appreciated that the monomers can be formed into a polymer microarray on the substrate surface using a range of techniques known in the art. In one embodiment of the present invention, the elements of the microarray are formed by depositing small drops of each monomer solution at discrete locations on the substrate surface, preferably by using an automated liquid handling device. As mentioned above, the monomers of the invention are initially provided as diluted liquids or solutions of dissolved solids. Once the stock solutions of the polymeric biomaterials have been prepared, a predetermined volume of each biomaterial stock solution is placed in the separate reservoirs of the robotic liquid handling device.

The drops may be deposited on the substrate surface using a microarray of pins (e.g., ChipMaker2™ pins, available from TeleChem International, Inc. of Sunnyvale, Calif.). A range of pins exist that take a sample volume up by capillary action and deposit a spot volume of 1 to 10 nl or more. These pins may be controlled by a robotic liquid handling device that controls the speed and travel pattern of the pins as well as automatic washing cycles and pauses between deposition steps. The device carrying the pins may be programmed to change the amount and length of washing cycles between deposition steps and adjust the speed with which the pins are transported from the monomer supply to the substrate at which the monomer is deposited. In addition, the path over which the pins are transported may be optimized.

In another embodiment, the drops may be deposited on the substrate surface using syringe pumps controlled by micro-solenoid ink-jet valves that deliver volumes greater than about 10 nl (e.g., using printheads based on the SYNQUAD™ technology, available from Cartesian Technologies, Inc. of Irvine, Calif.). Alternatively, the drops may be deposited on the substrate surface using piezoelectric ink-jet fluid technology that deposits smaller drops with volumes between about 0.1 and 1 nl (e.g., using the MICROJET™ printhead available from MicroFab Technologies, Inc. of Plano, Tex.). Alternative techniques may be employed to deposit smaller or larger drops. For example, pins may be pre-tapped to release a large drop and then tapped on the substrate to release a smaller drop, just as a paintbrush is tapped on the side of the can to remove excess paint and prevent messy drips on the painted surface. Where small drops are used, they should be polymerized shortly after deposition, before the solvent evaporates. For example, a portion of an array may be deposited and polymerized before deposition of a second portion of the array.

In one embodiment, the drops are arranged as a rectangular microarray on a glass slide. The size of the array may be determined by the user and will depend on the size of the elements of the array, the spacing between the elements and the size of the substrate surface. The rectangular microarray may, for example, be an 18×40, an 18×54 or a 22×64 microarray; however, smaller, larger and alternatively shaped microarrays (e.g., square, triangular, circular, elliptical, etc.) may be used. The shape of the microarray and the arrangement and spacing of polymer elements within it may depend on the analytical methods used to examine the arrayed polymers. For example, a particular sensor may require a specific shape or distribution of polymer elements. One skilled in the art will recognize that the use of robotic controls to move the pins enables any distribution and arrangement of spots regardless of symmetry. In one embodiment, two or more identical arrays are deposited alongside one another so that experiments on the polymers may be repeated.

In one embodiment of the invention, each element of the microarray is formed by depositing a single drop taken from one of the monomer stock solutions. In another embodiment, some or all of the elements are formed by depositing at least two drops taken from one of the monomer stock solutions. In yet another embodiment, some or all of the elements are formed by depositing at least two drops taken from at least two different monomer stock solutions. In an alternative embodiment, stock solutions of mixed monomers are prepared.

In one embodiment, the dimensions of the elements of the microarray are substantially the same; however, in certain embodiments of the present invention, the dimensions of the elements of the microarray may differ from one element to the next. The “vertical dimension”, as that term is used herein, means the vertical dimension of the element when viewed from a direction that is parallel to the substrate surface (i.e., from the side). The “horizontal dimension”, as that term is used herein, means the horizontal dimension of the element when viewed from a direction that is perpendicular to the substrate surface (i.e., from above).

The vertical dimensions of elements of the microarray of the present invention are such that each element may comprise hundreds or even thousands of layers of polymer molecules. When viewed from above or from the side, the elements may be circular, oblong, elliptical, square or rectangular. For example, the overall shape of the elements may be sphere-like or disk-like. In one embodiment, the drops are deposited at intervals that range from about 300 to about 1200 μm. In one embodiment, the drops are deposited at about 720 μm intervals; however, the drops may be deposited at smaller or larger intervals. The size and density of the elements depends on the application. Smaller elements, e.g., spaced at intervals of 1 μm or less, may be preferred for chemical analysis to further increase the number of compounds that can be analyzed in one batch. For example, 100 million elements, spaced at 0.1 μm intervals, can fit in an area of a square millimeter. In other embodiments, the array may have a density of one or fewer polymer elements per square centimeter. In general, the density, vertical dimension, and horizontal dimension of the elements will be optimized for the particular manufacturing technique and the variable being tested. In one embodiment, polymer arrays of 576 spots (24×24) are formed in triplicate on glass slides as arrays containing a total of 1728 spots.

In an exemplary embodiment of the invention, the elements of the microarray are deposited on the substrate surface as drops that range in volume from 0.1 to 100 nl. However, smaller and larger volumes may be deposited on the substrate surface. The ultimate dimensions of the drops depend on the application. For example, for cell attachment, the vertical dimension of the elements should be between about 50 and 500 μm, and the horizontal dimension of the deposited drops should be between 300 and 600 μm. The element should be large enough to minimize edge effects, but, for a single cell, the element may not need to be any larger than 10 μm across.

The drop volume and monomer viscosity may be adjusted so that the polymer element is thinner than 50 μm or even essentially flat. The primary limits on drop size are the ability to detect and deposit tiny drops. For some applications, it may be desirable to deposit drops as thin as a few 10s of nanometers. Microinjectors and robots can produce arrays of miniscule droplets, but the viscosity of the precursor must be carefully controlled to prevent clogging. Ink-jet printers may be used to reproducibly deposit drops of a specified size. In addition, the precursor should not polymerize before deposition and perhaps clog the dispenser. Thicker polymer elements may be produced by depositing a larger volume of precursor solution or by depositing several layers at each location. Bigger drops are easily deposited by e.g., using bigger pins (e.g., from TeleChem International, Inc., Sunnyvale, Calif.). Drop size may need to be optimized for a variety of factors, including the space required by seeded cells, the ability of the pins to handle a particular volume of monomer solution depending on factors such as the viscosity of the solution and the reproducibility of drop deposition, and the volatility of the monomer or any solvent.

After the monomer has been deposited on the surface, it is polymerized. In one embodiment, e.g., polymerization of diacrylates, the microarray is exposed to UV light, which initiates polymerization. If a chemical initiator is used, the microarray is exposed to conditions under which the initiator will start reacting with the monomer. Exemplary radical initiators that may be used with the invention include, but are not limited to, azobisisobutylnitrile (AIBN), 2,2-dimethoxy-2-phenyl-acetophenone (DPMA), benzoyl peroxide, acetyl peroxide, and lauryl peroxide. Redox and thermal initiators may also be exploited. For example, peroxides may be combined with a reducing agent such as Fe²⁺, Cr²⁺, V²⁺, Ti³⁺, Co²⁺, Cu⁺, and amines such as N,N-dialkylaniline. These initiators may be mixed with the monomer solutions and co-deposited. Because such initiators are often sensitive to temperature, they should be deposited at depressed temperatures. The temperature is then raised to start polymerization. A monomer that polymerizes in air should be deposited under nitrogen or argon and then exposed to air to start polymerization. One skilled in the art will recognize that a wide variety of initiators may be employed with the invention depending on the monomes being deposited. A plethora of initiators are available from companies such as Sigma and Polysciences. In one embodiment of the invention, once the complete microarray of elements has been deposited and polymerized, the polymer microarray is placed in an evacuated desiccator at about 25° C. for 12 to 48 hrs to remove any residual solvent. Alternatively, or additionally, the microarray may be washed to remove the solvent.

In one embodiment, the substrate surface or the array is modified after the polymer array has been deposited. Self assembled monolayer (SAM) systems may be chosen that react with the base layer but not with the various polymers. Alternatively, the polymer array may be deposited directly on the substrate and the uncovered surface modified afterwards using standard organosilane chemistry. For example, it is well known that washing PLGA in an acidic solution makes it more cytophilic. Both acid and base washes may be tested on other polymers. Alternatively or in addition, the spots may be mechanically roughened.

One aspect of the present invention involves the recognition that an endless variety of polymers can be obtained according to the present invention by varying the compositions of the stock solutions that are initially added to the robotic liquid handling device and/or by layering drops taken from these stock solutions in a series of sequential deposition steps. To produce bulk quantities of polymers would require large amounts of monomer and solvents which would then have to be disposed of properly. Small amounts of stock solutions of the desired monomers can be used for multiple tests, enabling a large number of monomers to be mixed in several different proportions in a single experiment. In addition, fewer stock solutions are required than to deposit polymerized polymers in the array.

The composition of the polymers themselves may be analyzed spectrophotometrically, for example, by fluorescence, infrared, or Raman spectroscopy.

Cell Seeding

In one embodiment of the present invention, a microarray of biocompatible polymers provided according to the invention may be seeded with cells. The invention is appropriate for use with a wide range of cell types and is not limited to any specific cell type. Examples of cell types that may be used include but are not limited to bone or cartilage forming cells such as chondrocytes and fibroblasts, other connective tissue cells such as epithelial and endothelial cells, cancer cells, hepatocytes, islet cells, smooth muscle cells, skeletal muscle cells, heart muscle cells, kidney cells, intestinal cells, other organ cells, lymphocytes, blood vessel cells, and stem cells such as or mesenchymal stem cells. For therapeutic applications, it is preferable to practice the invention with mammalian cells, and more preferably human cells. However, non-mammalian cells such as bacterial cells (e.g., E. coli), yeast cells (e.g., S. cerevisiae) and plant cells may also be used with the present invention.

Embryonic stem cells (ES) are also suited for use with the invention. Embryonic stem (ES) cells, including human ES (hES) cells, are a promising source for cell transplantation due to their unique ability to give rise to all somatic cell lineages when they undergo differentiation (Dushnik-Levinson, M., et al., “Embryogenesis in vitro: study of differentiation of embryonic stem cells,” Biol Neonate 67, 77-83 (1995); Thomson, J. A., et al., “Embryonic stem cell lines derived from human blastocysts,” Science 282, 1145-1147 (1998); Wobus, A. M., “Potential of embryonic stem cells,” Mol Aspects Med 22, 149-164 (2001); Stocum, D. L., “Stem cells in regenerative biology and medicine,” Wound Repair Regen 9, 429-442 (2001)). Differentiation of ES can be induced by removing the cells from their feeder layer and growing them in suspension, resulting in cellular aggregation and formation of embryoid bodies (EBs), in which successive differentiation steps occur (Itskovitz-Eldor, J., et al., “Differentiation of human embryonic stem cells into embryoid bodies compromising the three embryonic germ layers,” Mol Med 6, 88-95 (2000)). Several studies have shown that chemical cues provided directly by growth factors or indirectly by feeder cells can induce ES cell differentiation towards specific lineages (Johansson, B. M., et al., “Evidence for involvement of activin A and bone morphogenetic protein 4 in mammalian mesoderm and hematopietic development,” Mol Cell Biol 15, 141-151 (1995); Schuldiner, M., et al., “Effects of eight growth factors on the differentiation of cells derived from human embryonic stem cells,” Proc Natl Acad Sci USA 97, 11307-11312 (2000); Guan, K., et al., “Embryonic stem cell-derived neurogenesis. Retinoic acid induction and lineage selection of neuronal cells,” Cell Tissue Res 305, 171-176 (2001); Kaufman, D. S., et al., “Hematopoietic colony-forming cells derived from human embryonic stem cells,” Proc Natl Acad Sci USA 98, 10716-10721 (2001)). However, none of these studies succeeded in controlling differentiation of the ES cells to form complex tissues. In some cell types, physical cues including surface interactions, shear stress and mechanical strain have induced differentiation (Ito, Y., “Surface micropatterning to regulate cells functions,” Biomaterials 20, 2333-2342 (1999); Ballermann, B. J., et al., “Shear stress and the endothelium,” Kidney Int Suppl 67, S100-108 (1998); Carter, D. R., et al., “Mechanobiology of skeletal regeneration,” Clin Orthop, S41-55 (1998); Ingber, D. E., et al., “Mechanochemical switching between growth and differentiation during fibroblast growth factor-stimulated angiogenesis in vitro: role of extracellular matrix,” J Cell Biol 109, 317-330 (1989)). The invention provides a method of screening polymers for suitability as substrates for stem cells proliferation and differentiation.

The cells are first cultured in a suitable growth medium, as would be obvious to one of ordinary skill in the art. See, for example, Current Protocols in Cell Biology, Ed. by Bonifacino et al., John Wiley & Sons Inc., New York, N.Y., 2000 (incorporated herein by reference). A microarray of biocompatible polymers prepared as above is then placed in a suitable container (e.g., a 25 mm by 150 mm round suspension culture dish or a TEFLON™ trough) and incubated with a solution of the cultured cells. In one embodiment, the cells are present at a concentration that ranges from about 10,000 to 500,000 cells/cm³. Higher and lower cell concentrations may be used. For example, some applications may benefit from concentrations in the millions of cells per cubic centimeter. The incubation time and conditions (e.g., temperature, CO₂ and O₂ levels, growth medium, etc.) will depend on the nature of the cells that are under evaluation. For most cell types, the choice of conditions will be obvious to one skilled in the art. The incubation time should be sufficiently long to allow the cells to adhere to the elements of the polymeric biomaterial microarray. In one embodiment of the invention, the environmental conditions will need to be optimized in a series of screening experiments.

A growth factor may be added to the medium in which the cells are incubated with the polymer array. In one embodiment, parallel experiments are conducted with and without the growth factor to determine if the growth factor modifies the response of the cells to a particular polymer. For example, a cell type may proliferate on a particular polymer in the presence of a growth factor but not otherwise, or vice versa, or the growth factor may have no affect on cell proliferation. Exemplary growth factors that may be exploited for use with the invention include but are not limited to activin A (ACT), retinoic acid (RA), epidermal growth factor, bone morphogenetic protein, platelet derived growth factor, hepatocyte growth factor, insulin-like growth factors (IGF) I and II, hematopoietic growth factors, peptide growth factors, erythropoietin, interleukins, tumor necrosis factors, interferons, colony stimulating factors, heparin binding growth factor (HBGF), alpha or beta transforming growth factor (α- or β-TGF), fibroblastic growth factors, epidermal growth factor (EGF), vascular endothelium growth factor (VEGF), nerve growth factor (NGF) and muscle morphogenic factor (MMP).

Cell Screening

In a preferred embodiment of the invention, the cellular behavior of the seeded cells is assayed for each element of the microarray. The invention employs a wide range of cell-based assays that enable the investigation of a variety of aspects of cellular behavior. Exemplary cell-based assays are discussed in our commonly owned application U.S. Ser. No. 09/803,319, entitled “Uses and Methods of Making Microarrays of Polymeric Biomaterials,” the entire contents of which are incorporated herein by reference.

The cellular behaviors that can potentially be investigated according to the invention include but are not limited to cellular adhesion, proliferation, differentiation, metabolic behavior (e.g., activity level, metabolic state, DNA synthesis, apoptosis, contraction, mitosis, exocytosis, synthesis, endocytosis, migration), gene expression, protein expression, and the degree or amount of any of these. One may be interested in screening for polymeric biomaterials that promote or inhibit the adhesion of a given cell type. It is also desirable to understand whether certain materials are toxic to cells or accelerate apoptosis. Alternatively or additionally, one may be interested in screening for biocompatible polymers that enhance the proliferation of a given cell type. For example, biocompatible polymers that enhance the adhesion and proliferation of chondrocytes could be used as scaffolds in the preparation of engineered cartilage.

One may further be interested in screening for polymeric biomaterials that cause attached cells to differentiate or de-differentiate in a desirable way. More specifically, one may be interested in screening for polymeric biomaterials that promote or inhibit the expression of a given gene within a cell. For example, polymeric biomaterials that support differentiation of neural stem cells into glial cells or neurons may be useful as scaffolds in the regeneration of neural tissue. Different growth factors or growth media may be tested to enhance this effect. Alternatively, it may be desirable to characterize the influence of a polymer on a cell's interaction with other cells, viruses, small molecules, DNA, biomolecules, etc. The cell's interactions with a selection or library of chemicals may be evaluated by producing an array with one polymer on which a variety of small molecules, DNA, biomolecules, etc. are immobilized.

It will be appreciated that any of the cell-based assays known in the art may be used according to the present invention to screen for desirable interactions between the biocompatible polymers of the microarray and a given cell type. When they are assayed, the cells may be fixed or living. Preferred assays employ living cells and involve fluorescent or chemiluminescent indicators, most preferably fluorescent indicators. A variety of fixed and living cell-based assays that involve fluorescent and/or chemiluminescent indicators are known in the art. For a review of cell-based assays, see Current Protocols in Cell Biology, Ed. by Bonifacino et al., John Wiley & Sons Inc., New York, N.Y., 2000; Current Protocols in Molecular Biology, Ed. by Ausubel et al., John Wiley & Sons Inc., New York, N.Y., 2000; Current Protocols in Immunology, Ed. by Coligan et al., John Wiley & Sons Inc., New York, N.Y., 2000; Sundberg, Curr. Opin. Biotechnol. 11: 47, 2000; Stewart et al., Methods Cell Sci. 22: 67, 2000; and Gonzalez et al., Curr. Opin. Biotechnol. 9: 624, 1998; all of which are incorporated herein by reference.

Cell-based assays screen for interactions at the cellular level using cellular targets and are to be contrasted with molecular-based assays that screen for interactions at a molecular level using molecular targets. Although the sheer number of cellular components and the inherent complexity of cellular behavior can make the interpretation of cell-based assays somewhat complex, their scope, practical relevance and versatility is significantly greater than that of some of the simpler but more specific molecular assays. Indeed, by employing a cellular environment to screen for a given outcome (e.g., expression of a gene of interest) the experimenter does not require prior knowledge of the specifics of the interactions involved (e.g., the nature of the surface receptor or cytoplasmic cascade that triggers expression of the gene of interest). As a consequence, when used with an appropriate assay, the “black box” that is the cellular machinery can, amongst other things, dramatically simplify and shorten the screening process.

Various protein markers may be used to determine the type or behavior of cells seeded on the polymeric biomaterials. For example, cytokeratin is a marker for epidermal cells while desmin is a marker for muscle cells, and nestin and GFAP production may be used to identify cells that are differentiating as nerve cells. The presence of alpha feto protein may be used to confirm the differentiation of cells towards liver cells, and vimentin assays may be used to confirm that cells are differentiating as mesodermal cells. Actin indicates contractile activity in cells. Other markers may be used to identify expression of a predetermined gene, whether cells have fully differentiated, or whether there are still precursor cells seeded on the polymeric biomaterials.

Alternatively or in addition, genetic markers associated with particular cell types or cell behaviors may be used to characterize the seeded cells. For example, expression of the neurofilament heavy chain gene is associated with brain tissue, while expression of the alpha-1 anti-trypsin gene is associated with liver tissue. Other genetic markers are listed in Schuldiner, et al., PNAS, 97: 11307-11312, 2000, the entire contents of which are incorporated herein by reference.

It will be appreciated that any of the cell-based assays known in the art may be used according to the present invention to screen for desirable interactions between the polymeric biomaterials of the microarray and a given cell type. When they are assayed, the cells may be fixed or living. Preferred assays employ living cells and involve fluorescent or chemiluminescent indicators, most preferably fluorescent indicators. A variety of fixed and living cell-based assays that involve fluorescent and/or chemiluminescent indicators are known in the art. For a review of cell-based assays, see Current Protocols in Cell Biology, Ed. by Bonifacino et al., John Wiley & Sons Inc., New York, N.Y., 2000; Current Protocols in Molecular Biology, Ed. by Ausubel et al., John Wiley & Sons Inc., New York, N.Y., 2000; Current Protocols in Immunology, Ed. by Coligan et al., John Wiley & Sons Inc., New York, N.Y., 2000; Sundberg, Curr. Opin. Biotechnol. 11: 47, 2000; Stewart et al., Methods Cell Sci 22: 67, 2000; and Gonzalez et al., Curr. Opin. Biotechnol. 9: 624, 1998; all of which are incorporated herein by reference. Additional immunohistochemical and immunocytochemical methods are disclosed in Microscopy, Immunohistochemistry, and Antigen Retrieval Methods, by M. A. Hayat, Plenum Press, 2002 and Immunocytochemistry and in Situ Hybridization in the Biomedical Sciences, by Julian E. Beesley, Birkhauser Boston, 2000.

Specific cell-based assays that can be used according to the present invention include but are not limited to assays that involve the use of phase contrast microscopy alone or in combination with cell staining; immunocytochemistry with fluorescent-labeled antibodies; fluorescence in situ hybridization (FISH) of nucleic acids; gene expression assays that involve fused promoter/reporter sequences that encode fluorescent or chemiluminescent reporter proteins; in situ PCR with fluorescently labeled oligonucleotide primers; fluorescence resonance energy transfer (FRET) based assays that probe the proximity of two or more molecular labels; and fused gene assays that enable the cellular localization of a protein of interest. The steps involved in performing such cell-based assays are well known in the art. For the purposes of clarification only, and not for limitation, certain properties and practical aspects of some of these cell-based assays are considered in greater detail in the following paragraphs.

Currently, fluorescence immunocytochemistry combined with fluorescence microscopy allows researchers to visualize biological moieties such as proteins or DNA within a cell (for a review on confocal microscopy, see Mongan et al., Methods Mol. Biol. 114: 51, 1999; for a review on fluorescence correlated spectroscopy, see Rigler, J. Biotechnol. 41: 177, 1995; and for a review on fluorescence microscopy, see Hasek et al., Methods Mol. Biol. 53: 391, 1996; all of which are incorporated herein by reference). One method of fluorescence immunocytochemistry involves the first step of hybridizing primary antibodies to the desired cellular target. Then, secondary antibodies conjugated with fluorescent dyes and targeted to the primary antibodies are used to tag the complex. The complex is visualized by exciting the dyes with a wavelength of light matched to the dye's excitation spectrum. A variety of fluorescent dyes such as fluorescein and rhodamine are known in the art. Appropriate antibodies are well described in the art, and a variety of labeled and unlabeled primary and secondary antibodies are available commercially (e.g., from Sigma).

Colocalization of biological moieties in a cell may be performed using different sets of antibodies for each cellular target. For example, one cellular component can be targeted with a mouse monoclonal antibody and another component with a rabbit polyclonal antibody. These are designated as primary antibodies. Subsequently, secondary antibodies to the mouse antibody or the rabbit antibody, conjugated to different fluorescent dyes having different emission wavelengths, are used to visualize the cellular target. An ideal combination of dyes for labeling multiple components within a cell would have well-resolved emission spectra. In addition, it would be desirable for this combination of dyes to have strong absorption at a coincident excitation wavelength.

As will be appreciated by one of ordinary skill in the art, fluorescent immunocytochemistry can be used to assay for cellular adhesion, gene expression, and cell proliferation. In one embodiment, fluorescent molecules such as the Hoechst dyes (e.g., benzoxanthene yellow or DAPI (4,6-diamidino-2-phenylindole)) that target and stain DNA directly and non-specifically can be used to estimate the total cell population on each element of a seeded microarray of the invention. As is well known in the art, such estimates can be used to normalize the measured levels of a biological moiety of interest (e.g., an expressed protein) within the cells that are attached to the elements of a seeded microarray.

Fluorescence in situ hybridization (FISH) typically involves the fluorescent tagging of an oligonucleotide probe to detect a specific complementary DNA or RNA sequence. For a review of FISH see, Swiger et al., Environ. Mol. Mutagen. 27: 245, 1996; Raap, Mut. Res. 400: 287, 1998; and Nath et al., Biotechnic. Histol. 73: 6, 1997; all of which are incorporated herein by reference. An alternative approach is to use an oligonucleotide probe conjugated with an antigen such as biotin or digoxygenin and a fluorescently tagged antibody directed toward that antigen to visualize the hybridization of the probe to its DNA target. A variety of FISH formats are known in the art. See, for example, Dewald et al., Bone Marrow Transplant. 12: 149, 1993; Ward et al., Am. J. Hum. Genet. 52: 854, 1993; Jalal et al., Mayo Clin. Proc. 73: 132, 1998; Zahed et al., Prenat. Diagn. 12: 483, 1992; Kitadai et al., Clin. Cancer Res. 1: 1095, 1995; Neuhaus et al., Human Pathol. 30: 81, 1999; Buno et al., Blood 92: 2315, 1998; Patterson et al., Science 260: 976, 1993; Patterson et al., Cytometry 31: 265, 1993; Borzi et al., J. Immunol. Meth. 193: 167, 1996; Wachtel et al., Prenat. Diagn. 18: 455, 1998; Bianchi, J. Perinat. Med. 26: 175, 1998; and Munne, Mol. Hum. Reprod. 4: 863, 1998; all of which are incorporated herein by reference.

Fluorescence resonance energy transfer (FRET) provides a method for detecting the proximity of two or more biological compounds by detecting the long-range resonance energy transfer that can occur between two organic fluorescent dyes if the spacing between them is less than approximately 100 Å. Conversely, this effect can be used to determine that two or more biological compounds are not in proximity to each other. For reviews on FRET, see Clegg, Curr. Opin. Biotechnol. 6: 103, 1995; Clegg, Methods Enzymol. 211: 353, 1992; and Wu et al., Anal Biochem. 218: 1, 1994; all of which are incorporated herein by reference.

Cell-based assays that use promoter/reporter genes are designed to assay for expression of a gene of interest. Typically, this is achieved by transforming a given cell type with a plasmid comprising the promoter region of the gene of interest fused to the reporter sequence of a fluorescent or chemiluminescent protein. If the cytoplasmic cascade that normally leads to expression of the gene of interest and involves binding of a promoter moiety to the promoter sequence of the gene of interest is triggered, the transformed cells will begin to produce the reporter protein. Reporter genes that are known in the art include the genes that code for the family of blue, cyan, green, yellow, and red fluorescent proteins; the gene that codes for luciferase, a protein that emits light in the presence of the substrate luciferin; and the genes that code for β-galactosidase and β-glucuronidase (proteins that hydrolyze colorless galactosides and glucuronides respectively to yield colored products). A variety of vectors that contain fused promoter/reporter genes are available commercially (e.g., from Clontech Laboratories, Inc. of Palo Alto, Calif.).

In another embodiment, an automated device may be used to analyze the cell-based assays for each element of the polymeric biomaterial microarray. The devices may be manually or automatically operated. For example, an automated device that detects multicolored luminescent indicators can be used to acquire an image of the microarray and resolve it spectrally. Without limiting the scope of the invention, the device can detect samples by imaging or scanning. Imaging is preferred since it is faster than scanning. Imaging involves capturing the complete fluorescent or chemiluminescent data in its entirety. Collecting fluorescent or chemiluminescent data by scanning involves moving the sample relative to the imaging device.

An exemplary device may include three parts: 1) a light source, 2) a monochromator to spectrally resolve the image, or a set of narrow band filters, and 3) a detector array. The light source is only required for the detection of fluorescent indicators. In one embodiment, the light source may be derived from the output of a white light source such as a xenon lamp or a deuterium lamp that is passed through a monochromator to extract out the desired wavelengths. Alternatively, filters could be used to extract the desired wavelengths. In another embodiment, any number of continuous wave gas lasers can be used. These include, but are not limited to, any of the argon ion laser lines (e.g., 457, 488, 514 nm, etc.), a HeCd laser, or a HeNe laser. Furthermore, solid state diode lasers could be used.

To spectrally resolve two different fluorescent or chemiluminescent indicators, light from the microarray may be passed through an image-subtracting double monochromator. Alternatively, the fluorescent or chemiluminescent light from the microarray may be passed through two single monochromators with the second one reversed from the first. The double monochromator consists of two gratings or two prisms and a slit between the two gratings. The first grating spreads the colors spatially. The slit selects a small band of colors, and the second grating recreates the image.

The fluorescent or chemiluminescent images may be recorded using a camera fitted with a charge-coupled device (CCD). A CCD is a light sensitive silicon solid state device composed of many small pixels. The light falling on a pixel is converted into a charge pulse which is then measured by the CCD electronics and represented by a number. A digital image is the collection of such light intensity numbers for all of the pixels from the CCD. A computer can reconstruct the image by varying the light intensity for each spot on the computer monitor in the proper order. As is well known in the art, such digital images can be stored on disk, transmitted over a computer network and analyzed using powerful image processing techniques. Any two-dimensional detector or CCD can be used. A variety of CCDs and two-dimensional detectors are available commercially (e.g., from Hamamatsu Corp. of Bridgewater, N.J.). A variety of automated imaging systems that combine CCDs with computers and image processing software are also available commercially (e.g., the ARRAYWORXS™ microarray scanner available from Applied Precision, Inc. of Issaquah, Wash.).

In one embodiment, the fluorescent or chemiluminescent light is detected by scanning the microarray of the present invention. An apparatus using the scanning method of detection collects light data from the sample relative to a detection device by moving either the microarray or the detection device. For example, the microarray may be scanned by moving the detection device. When two different fluorescent or chemiluminescent indicators need to be resolved, the light from the microarray may be passed thought a single monochromator, a grating or a prism. Alternatively, filters could be used to resolve the colors spectrally. For the scanning method of detection, the detector is preferably a diode array which records the light that is emitted at a particular spatial position. As is well known in the art, software can then be used to recreate the scanned image, resulting in a single image containing the entire microarray of the invention. As described above, such digital images can be stored on disk, transmitted over a computer network and analyzed using very powerful image processing techniques.

Cell-Polymer Interactions

The methods described above provide a system for the examination of polymer affects on cell gene expression, differentiation, and other aspects of cell metabolism. The polymer arrays described above may be produced in large quantities quite reproduceably. These arrays may be tested with various cell types or under various conditions, including the presence or absence of various growth factors. This enables the rapid testing of polymer libraries with many cell types under varying conditions. In addition, it allows identification of polymers that permit varying levels of cell growth and proliferation, permit cell-type specific growth, and permit growth factor-specific proliferation and differentiation. Polymers and growth factors and polymer growth factor combinations may be identified that promote a specific level of cell activity. For example, a particular monomer may facilitate one level of activity when co-polymerized with monomer A and a different level of activity when co-polymerized with monomer B.

In one embodiment, the invention may be used to identify polymer-growth factor combinations that promote particular differentiation pathways. For example, a particular polymer in combination with retinoic acid may promote differentiation of stem cells into epithelial-like cells. Substitution of a different growth factor, or a different polymer, may induce the stem cells to follow a different path.

The polymer arrays of the invention may be more finely tuned by the addition of cell membrane components, adhesion peptides, or other materials. These materials may be used to promote differentiation along a particular path or to prevent de-differentiation of cells such as chondrocytes that are particularly prone to de-differentiation.

### EXAMPLES

### Example 1

### Production of a Polymer Array

The use of robotic fluid handling for the production of DNA, protein, and small molecule microarrays is well defined (G. MacBeath, et al., Journal of the American Chemical Society 121, 7967-7968 (1999); G. MacBeath, et al., Science 289, 1760-1763 (2000); M. Schena, et al., Science 270, 467-470 (1995)). However, the deposition of structurally diverse acrylate monomers to produce a uniform, cell-compatible polymer microarray required significant modification of existing robotic technology. First, some acrylate monomers are viscous, affecting all aspects of monomer printing including pre-printing pin priming, fluid ejection at printing, and pin washing. Another problem unique to these arrays is that the ordinary sensitivity of radical polymerization to oxygen inhibition is particularly evident at small volumes. Consequently, we performed our printing in an atmosphere of humid argon with oxygen present at less than 0.1%. Humidity helps minimize failed printing, presumably by reducing static effects. Finally, some monomers spread soon after deposition, forming irregular polymer spots, while others started to evaporate a few minutes after deposition. To address these issues our robot was modified by inclusion of a long wave UV lamp which immediately polymerized the monomers following each round of monomer deposition.

Epoxy coated glass slides (Xenopore, Hawthorne, N.J.) were dip coated into 4% (w/v) poly(hydroxyethyl methacrylate) (pHEMA, Aldrich, Milwaukee, Wis.) solution in ethanol and dried for 3 days prior to use. Monomers (FIG. 2A) were purchased from Aldrich, Scientific Polymers (Onterio, N.Y.), and Polysciences (Warrington, Pa.). Stock solutions were prepared at a ratio of (v/v) 75% monomer, 25% DMF, and 1% (w/v) DPMA. These were then mixed pair-wise in 384 well black polypropylene plates at a ratio of 70:30 (v/v). Monomers were mixed in all possible combinations with the exception of monomer 17, which was substituted with monomer 25 to increase polymer hydrophilicity.

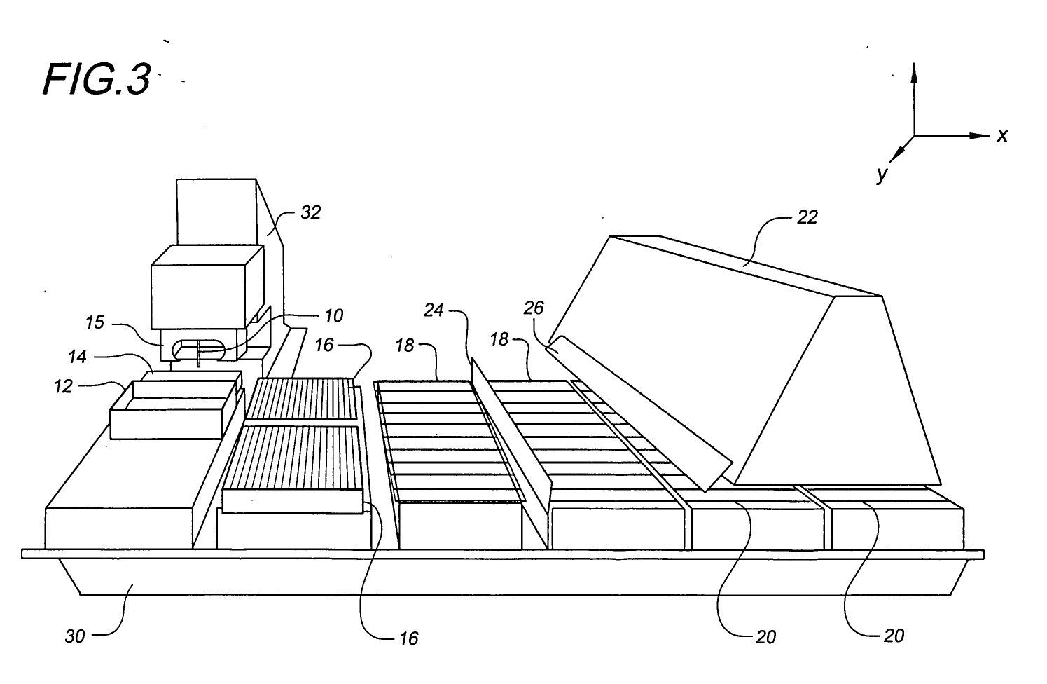

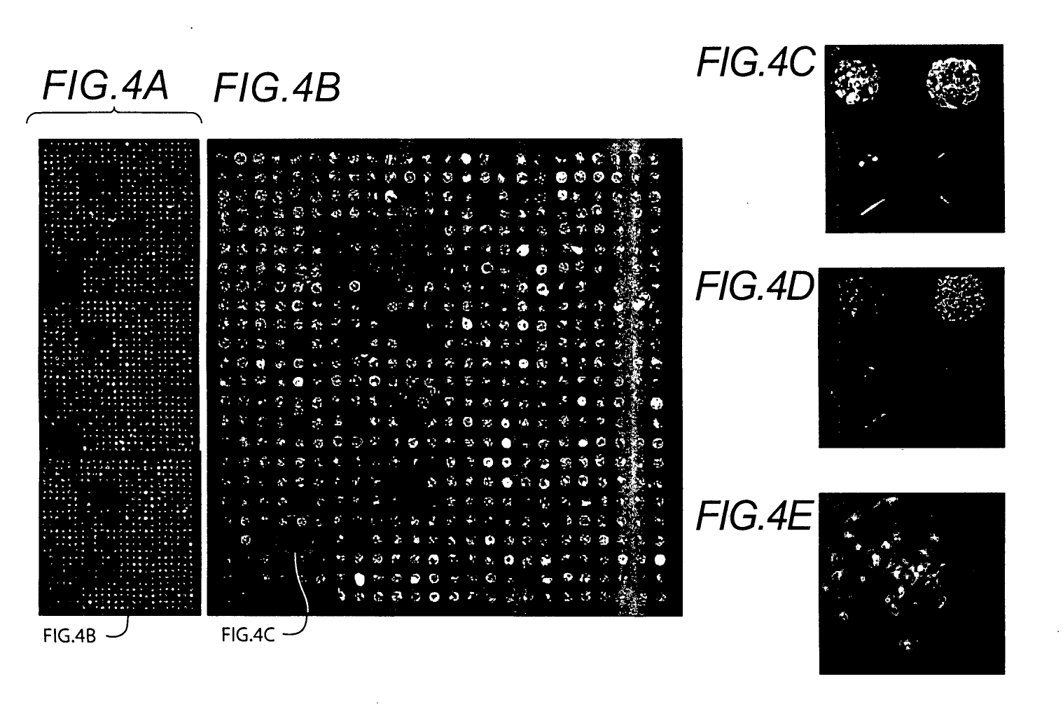

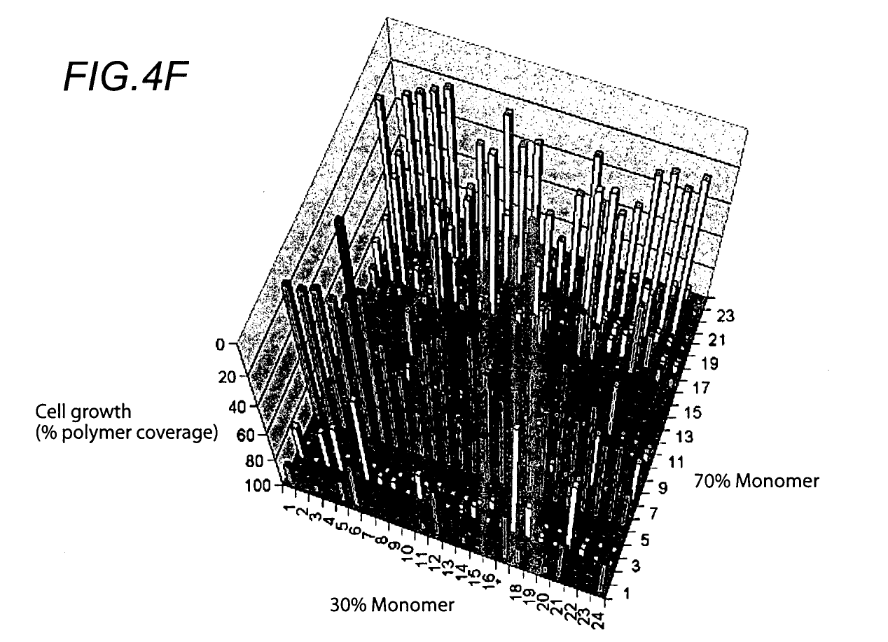

Monomers were printed using CMP9B or CMP6B pins (Telechem International, Sunnyvale, Calif.) with a Pixsys 5500 robot (Cartesian, Ann Arbor, Mich.) in humid argon. Printing of acrylate monomers required several modifications to existing printing methods: 1) incorporation of 25% dimethyl formamide to reduce viscosity, 2) substantially increasing washing and preprinting steps, and 3) modification of pin speed and size. FIG. 3 shows an exemplary apparatus for producing arrays for use with the invention. Pins 10 were initially washed in DMF in reservoir 12 with agitation for about 10 seconds, and placed in a vacuum apparatus 14 to remove the DMF. Four pins 10 were used, but the block 15 that retains the pins can hold 32. The receptacles for the unused 28 pins in the vacuum were easily stopped with tape to decrease the pressure in the vacuum. The pins 10 were dipped in the appropriate monomer solutions in tray 16 for about 3 seconds and tapped on a slide in row 18 to remove excess monomer solution. Pins 10 were tapped multiple times (20-30 times) using multiple tapping sites to remove excess from the pins until there was sufficient solution on the pin to deposit reproducibly. The pins were then translated to the slides in array 20 on which the arrays were produced and allowed to deposit monomer on each slide. The slides in array 20 were transferred under a UV lamp 22 and the pins were rinsed for about 10 s. A barrier 24 between the lamp and the monomer reservoir 16 and a baffle 26 attached to the housing of UV lamp 22 prevented the monomer from polymerizing in the reservoir. The process was then repeated, starting with the initial washing step. The table 30 translates along the x axis, and the robot arm 32 translates the pins along the x and y axes.

To facilitate analysis, all 24 polymers composed of 70% of a particular monomer were produced as a 6×4 group on the array, as highlighted by the red and yellow boxes (FIG. 2C). Three blocks of 576 polymers were produced on each slide, with a center-to-center spacing of 740 microns (FIG. 2B). After each round of printing on 10 slides, the slides were polymerized by exposure to longwave UV (UVP Blak-Ray, Upland Mich.) for ˜10 seconds. The monomers polymerized into rigid polymer spots which were firmly attached to the slide. While the vast majority of polymers remained attached to the matrix during analysis, certain particularly hydrophilic polymers (composed of 30% monomer 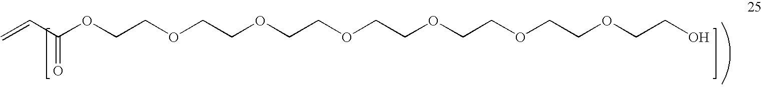
did fall off after extensive submersion. After the chips were printed, they were dried at <50 mTorr for at least 7 days. Chips were sterilized by exposure to UV for 30 minutes on each side, and then washed with PBS and medium for 30 minutes prior to use. (FIG. 2C, D).

### Example 2

### Cell Culture

H9 cells (Thomson, J. A., et al., “Embryonic stem cells lines derived from human blastocysts”, Science 282, 1145-1147 (1998)) were grown as described in Spradling, A., et al., “Stem cells find their niche”, Nature 414, 98-104 (2001), the entire contents of which are incorporated herein by reference. C2C12 cells were grown as described in Yaffee, D. & Saxel, O., “Serial passaging and differentiation of myogenic cells isolated from dystrophic mouse muscle”, Nature 270, 725-7 (1977). Specifically, hES cells (H9 clone) were grown on mouse embryo fibroblasts (Cell Essential) in KnockOut Medium (Gibco-BRL, Gaithersburg, Md.), a modified version of Dulbeco's modified Eagle's medium optimized for ES cells (Itskovitz-Eldor, et. al., (2000) Mol. Med. 6, 88-95, the contents of which are incorporated herein by reference). Tissue cover plates were covered with 0.1% gelatin (Sigma). Culture were grown in 5% CO₂ and were routinely passaged every 5-6 days after disaggregating with 1 mg/ml collagenase type IV (Gibco-BRL). To induce formation of EBs, hES colonies were digested using either 1 mg/ml collagenase type IV or trypsin/EDTA (0.1%/1 mM) and transferred to petri dishes to allow their aggregation and prevent adherence to the plate. Embryoid bodies were trypsinized after 6 days according to Levenberg, S., et al., “Differentiation of Human Embryonic Stem Cells on Three Dimensional Polymer Scaffold”, Proc. Nat. Acad. Sci., 100: 12741-12746 (2003). Specifically, EB's were dissociated with 0.025%/0.01% trypsin/EDTA and washed with PBS containing 5% FBS. Cells were added to the growth media (KO DMEM, 20% heat inactivated fetal bovine serum, L-Glutamine, B-Mercaptoethanol, minimal essential amino acids (Invitrogen, Carlsbad, Calif.), and 1 μM retinoic acid (Aldrich) when indicated), and then seeded onto chips in 26×100 mm Teflon dishes. Chips were incubated at 37° C. with 5% CO₂ and media was changed after 1 day, and then every 2 days thereafter.

### Example 3

### Immunohistochemistry

Chips were washed, fixed in 4% paraformaldehyde for 8 minutes, blocked with 10% goat serum (Zymed, San Francisco, Calif.) and permeablized with 0.2% triton X-100 for 30 minutes. Primary antibodies, Ms anti-Cytokeratin 7, Ms anti-Myogenin (Dako, Carpinteria, Calif.), Rb anti-Vimentin (Biomeda, Foster City, Calif.) in PBS with 3% goat serum were incubated on the chips for 1 hr. Chips were washed 3 times in 1% goat serum PBS. A mixture of Goat anti-Ms Alexa 555, Goat anti Rb Alexa, and SytoX24 (Molecular Probes, Eugene, Oreg.) were diluted into 3% goat serum PBS and incubated on the chips for 1 hr. Slides were washed 3 times in 1% goat serum PBS and dipped in 0.5 mM Tris Cl pH 7.5 to remove salt, and air dried immediately prior to scanning. Slides were then scanned using an Arrayworx autoloader scanner (API, Issaquah, Wash.) (FIG. 2).

### Example 4

### Evaluation of Cell-Polymer Interactions of hES Cells

A large variety of acrylate-based polymers have been used for tissue engineering, surgical glues, and drug delivery (Stocum, D. L., “Stem cells in regenerative biology and medicine”, Wound Repair Regen 9, 429-442 (2001)). There are a diverse collection of monomers commercially available, and these can be polymerized quickly using a light-activated radical initiator. To maximize throughput and minimize use of expensive reagents and cells, we developed a cell-compatible, miniaturized, polymer array. Using a modified fluid handling robot, we deposited 576 different combinations of 25 different acrylate, diacrylate, dimethacrylate, and triacrylate monomers in triplicate onto a poly(hydroxyethyl methacrylate) (pHEMA) coated slide (see FIG. 2). pHEMA has been known to effectively inhibit cell growth (Itskovitz-Eldor, J., et al., “Differentiation of human embryonic stem cells into embryoid bodies compromising the three embryonic germ layers”, Mol Med 6, 88-95 (2000)). After each round of deposition, the monomers were polymerized by brief exposure to long wave UV light. The synthesis of polymers in arrayed form onto a conventional 25×75 mm glass slide allows for easy, simultaneous staining and four-color fluorescence imaging of multiple slides, each containing 1,728 individual polymer spots with 20, 1728 spot polymer arrays being synthesized in a single day (FIG. 2).

To identify materials that could enable new levels of control over hES cell behavior, we tested the polymer arrays for their affects on the attachment, proliferation, and gene expression of hES cells. To initiate differentiation, embryoid bodies (EB) were allowed to form for 6 days. These were then trypsinized and 6 million cells seeded onto the arrays. The cells were incubated with the growth factor retinoic acid (RA) on the arrays for 6 days. Arrays were then fixed and stained for 1) cytokeratin 7, an intermediate filament protein found in most glandular and transitional epithelia (Johansson, B. M., et al., “Evidence for involvement of activin A and bone morphogenetic protein 4 in mammalian mesoderm and hematopoietic development”, Mol Cell Biol 15, 141-151 (1995)), 2) vimentin, an intermediate filament protein common in many cells of mesenchymal origin and 3) DNA/Nucleus with SYTO 24 (Molecular Probes, Eugene, Oreg.) (FIG. 2).

In general, cell growth is supported on the majority of these materials (FIG. 2F). However, certain monomers inhibit hES cell growth, in particular, polymers containing monomers 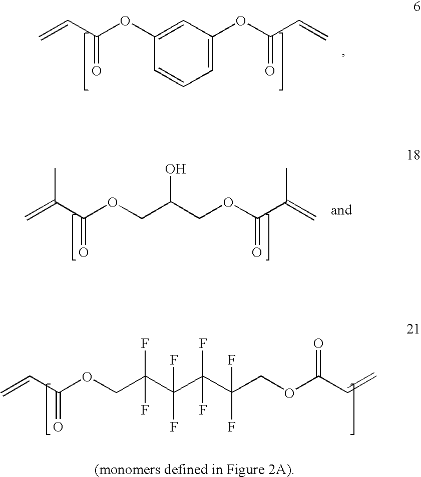
Interestingly, the inhibitory effects of certain monomers can be masked by the presence of other monomers. For example, polymers composed of 30% monomer 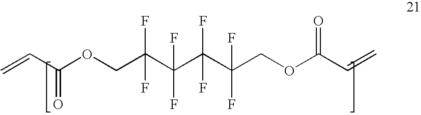
support growth when the other 70% is monomer 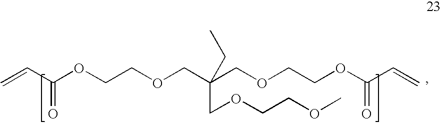
but significantly inhibit growth with 70% monomer 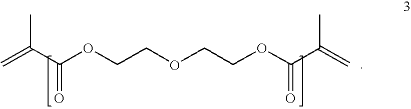
The majority of polymers supporting growth also allow for differentiation into cytokeratin-7 positive cells (FIG. 2). This simple, one-step production of cytokeratin positive cells could potentially be a useful method for the production of epithelia for tissue engineering and cell therapy. To our knowledge this is the first description of an efficient method for enrichment of epithelial-like cells from hES cells.

### Example 5

### Focus on hES Cells and Favorable Polymers

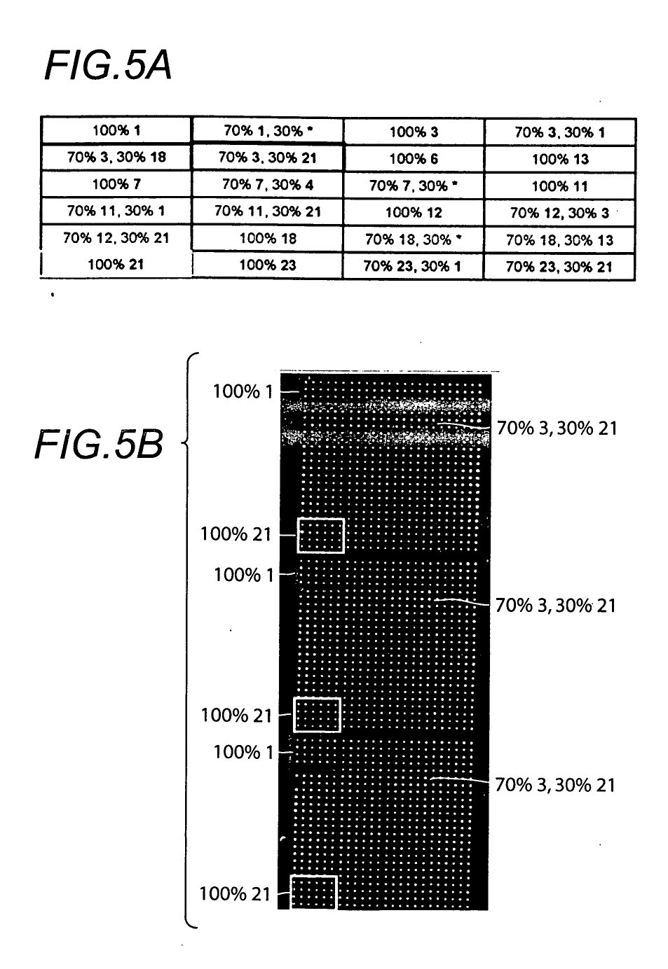

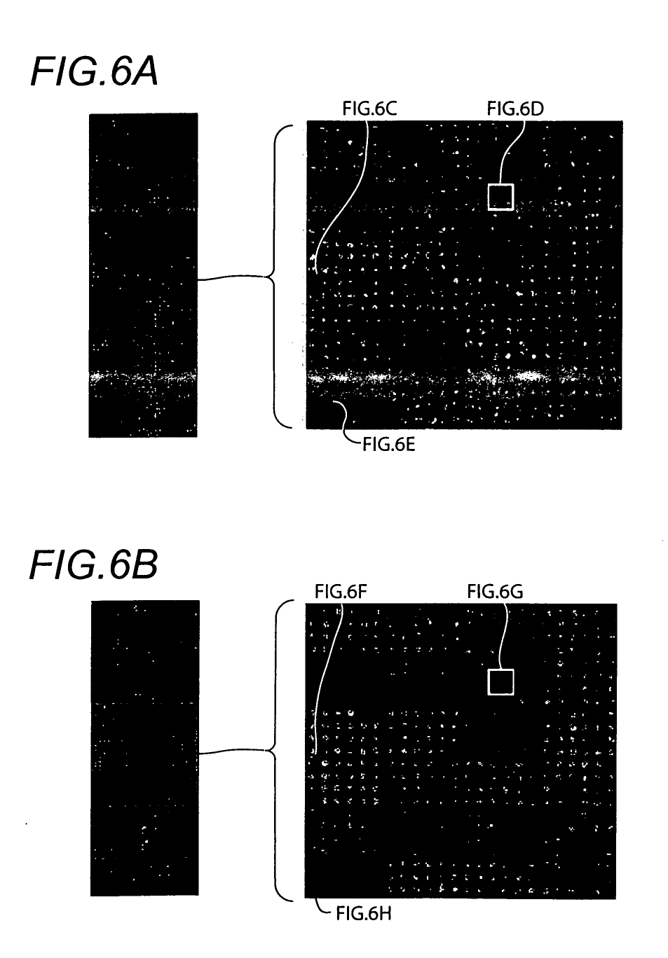

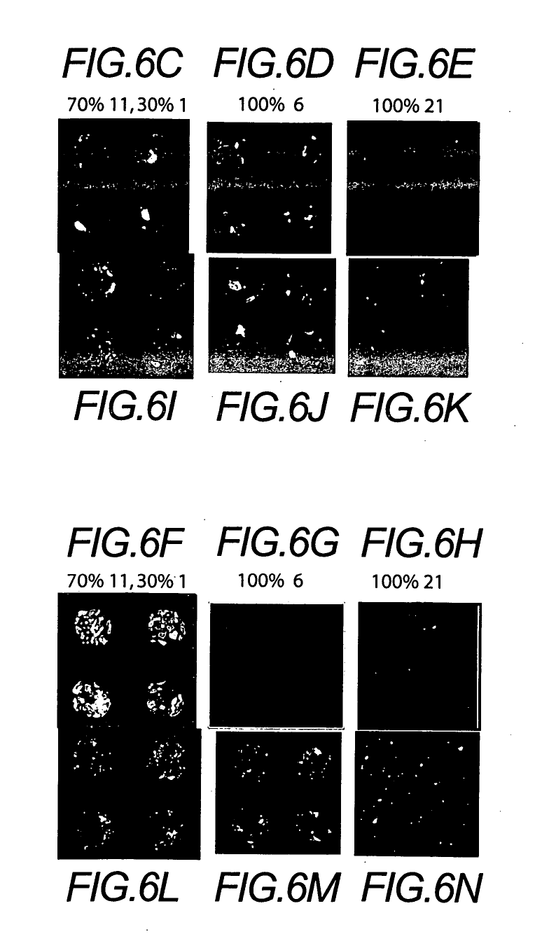

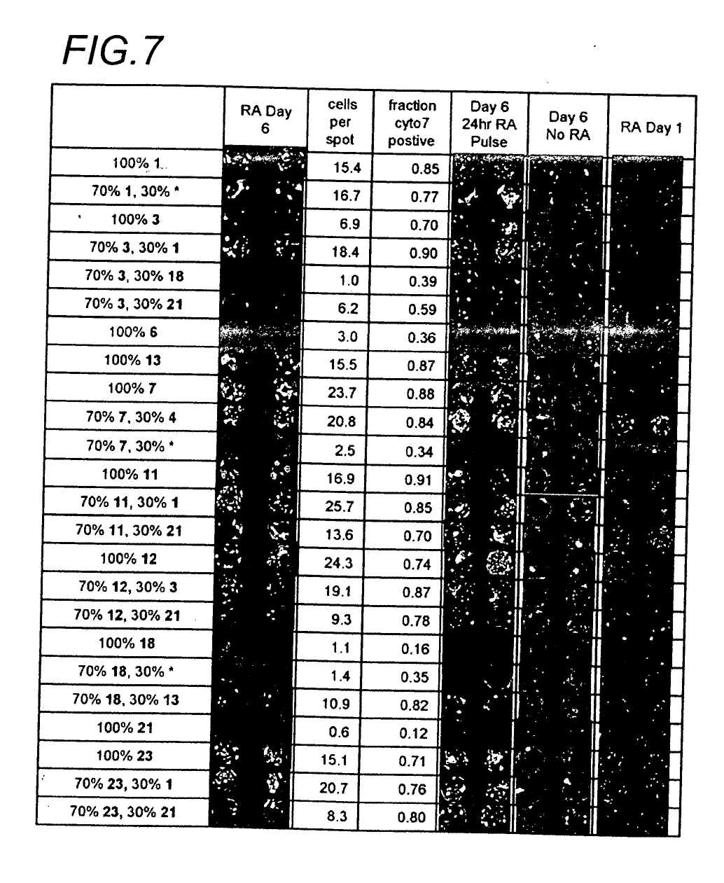

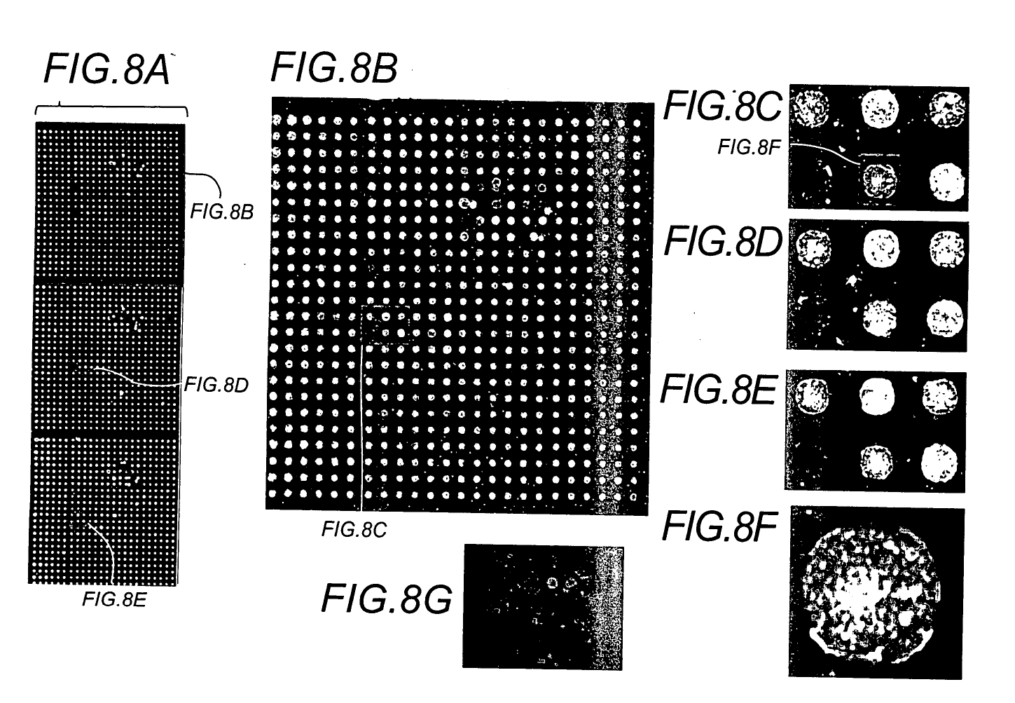

To more thoroughly study polymers of interest and their effects on hES differentiation we created polymer arrays with 24 polymers of interest identified in the first screen (FIG. 5). Each “hit” array contained 1,728 polymer spots; 24 polymers materials with 72 replicates per array. These were seeded with fewer cells, only 4 million, to more clearly identify polymer effects. Both soluble factors, such as growth factors, and the matrix on which they grow have the potential to affect cell behavior (Dushnik-Levinson, M., et al., “Embryogenesis in vitro: study of differentiation of embryonic stem cells”, Biol Neonate 67, 77-83 (1995); Thomson, J. A., et al., “Embryonic stem cell lines derived from human blastocysts”, Science 282, 1145-1147 (1998)). To more carefully examine the interplay of polymer and growth factor effects on cell behavior, arrays were tested with the growth factor RA, without RA, and with a 24 hour pulse of RA (FIGS. 6 and 7). Arrays were stained after 1 and 6 days.

The absence of retinoic acid has several key effects on cell behavior after six days: 1) much less expression of cytokeratin 7 was evident, and vimentin was generally upregulated, 2) cells were smaller and more tightly packed. Analysis of growth after one day (FIG. 6I-N) reveals that the presence of retinoic acid has, in general, little effect after 24 hours (I,L—monomer ratios 70% 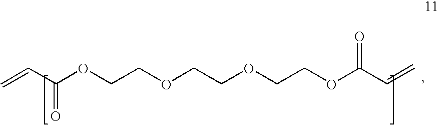
30% 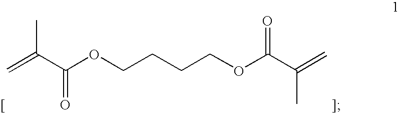
J,M—100% 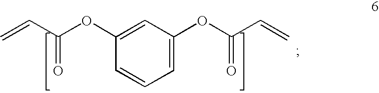
K,N—100% 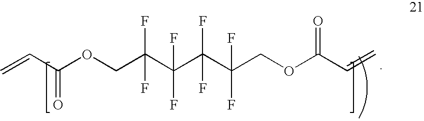
Surprisingly, some polymers only support growth when retinoic acid is absent. For example, cells are able to attach to polymers such as 100% 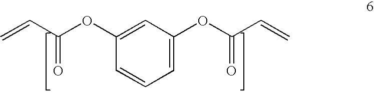
in similar quantities per spot with or without retinoic acid, as measured by cell counts after 24 hours of growth (FIGS. 6J, M). However after six days, 100% 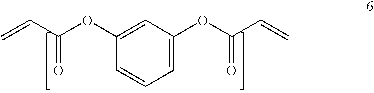
does not support proliferation of these cells in the presence of retinoic acid (FIG. 6D, G). In contrast, some polymers support growth in both conditions (e.g. 70% 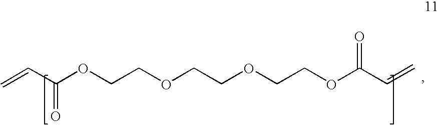
30% 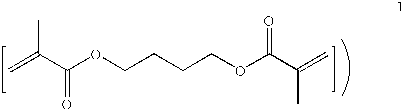
(FIGS. 6C, F), and others do not support growth in either (e.g. 100% 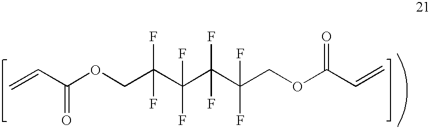
(FIGS. 6E, H). The discovery of polymers that support cell proliferation in a growth factor dependent manner could provide a new tool for controlling hES growth and proliferation.

To better understand the effects of these polymers on gene expression, cytokeratin 7 positive cells and total cells per spot were counted. After 6 days in the presence of RA, certain polymers, such as 100% 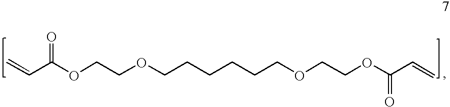
are nearly completely covered by cells, and have over 80% of the cells cytokeratin 7 positive (FIG. 7). In contrast, materials such as 100% 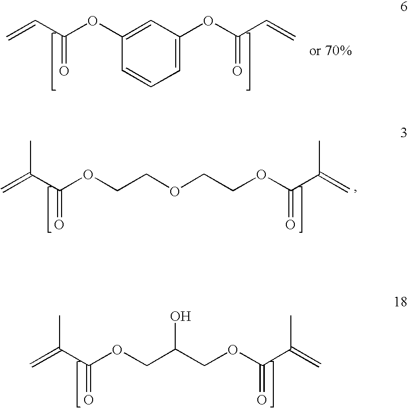
that show poor growth also have fewer than 40% cytokeratin 7 positive cells (FIG. 7). This difference is not apparent after 24 hours, suggesting proliferation of cytokeratin 7 positive cells on these polymers is inhibited to a greater extent than cytokeratin 7 negative cells. Analysis of the cell behavior on the hit arrays reveals a range of hES and differentiation activities in the presence and absence of RA on these materials (FIG. 7). This ranges from cell growth that completely covers the polymer spots (e.g. 100% 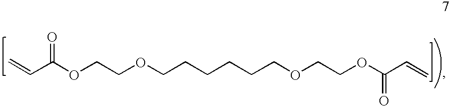
to weak cell growth (e.g. 70% 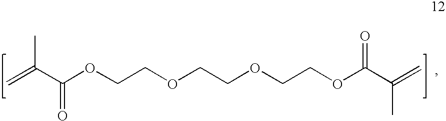
30% 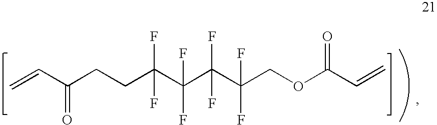
to essentially no growth (E.G. 100%

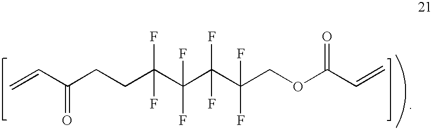

### Example 6

### C2C12-Polymer Interactions

To examine the whether polymer effects on cell growth are observed in other cell types, arrays in which monomer 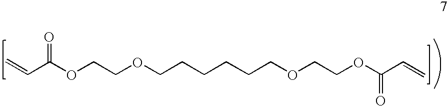
was replaced with 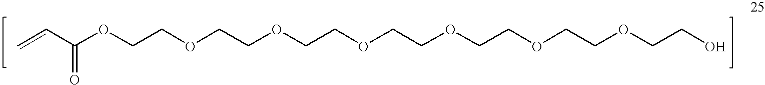
were seeded with 1 million C2C12 cells, an embryonic muscle cell line. Arrays were formed in trplicate. Unlike for the hES cells, almost all of the materials, including those containing 70% 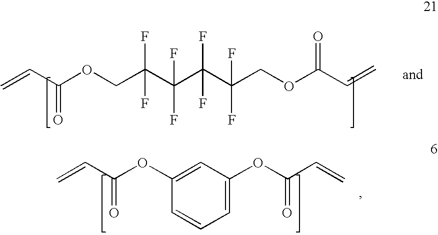
support the growth of these cells (FIG. 8). The mechanism behind these cell specific differences is unclear, but the identification of materials that selectively support the growth of specific cell types may be exploited to create complex tissue engineered constructs in which different polymers support different cells to conduct fundamental studies using multiple cell types.

### Example 7

### Differentiation of hES Cells in the Presence of Retinoic Acid

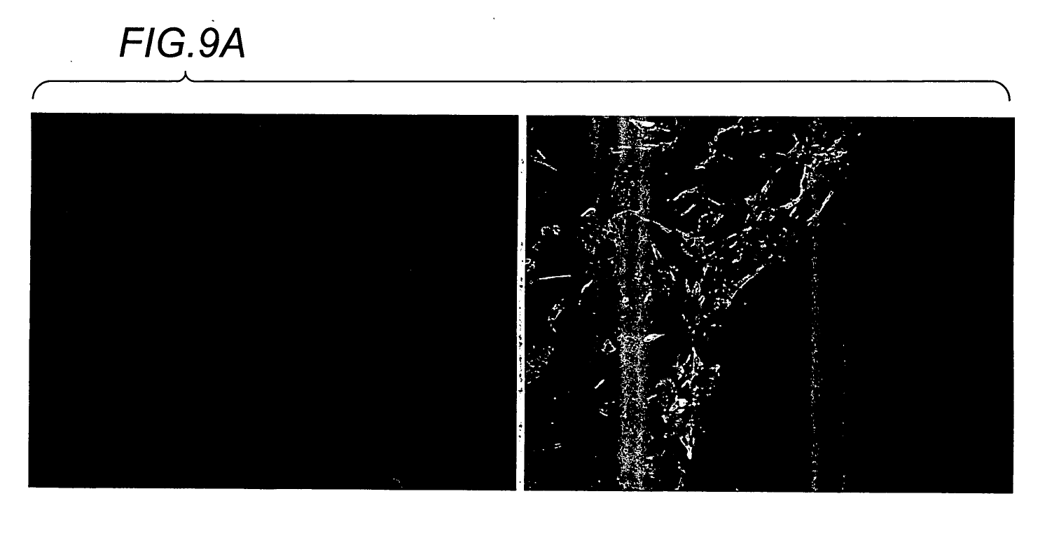

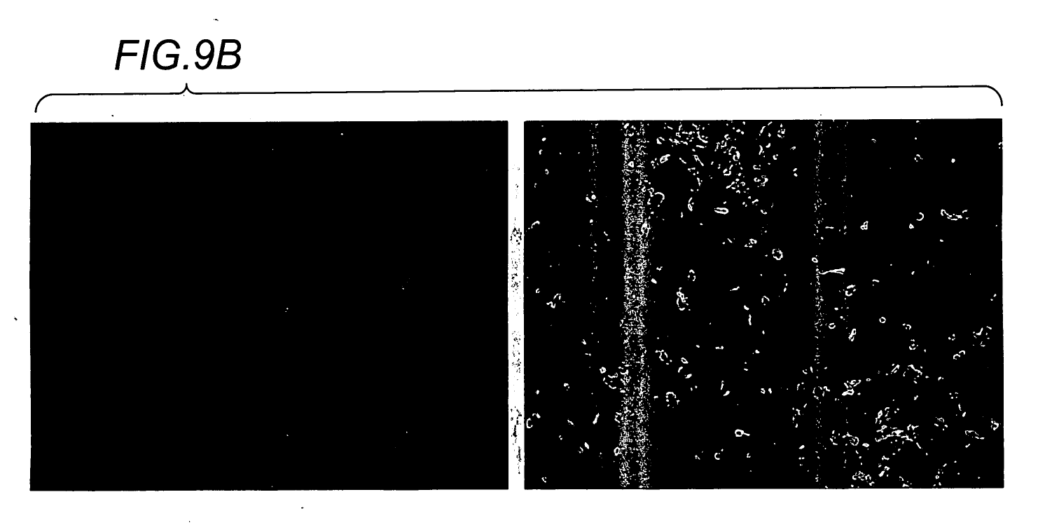

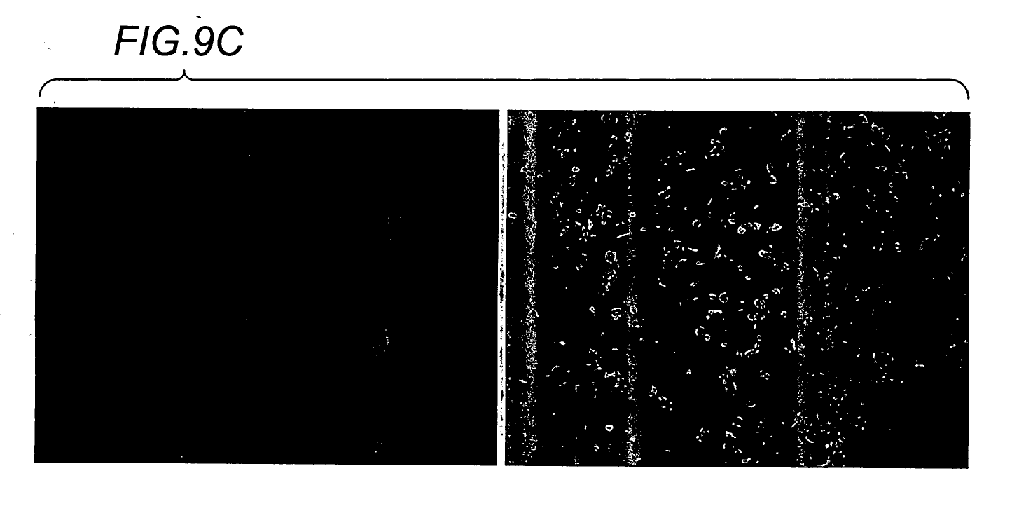

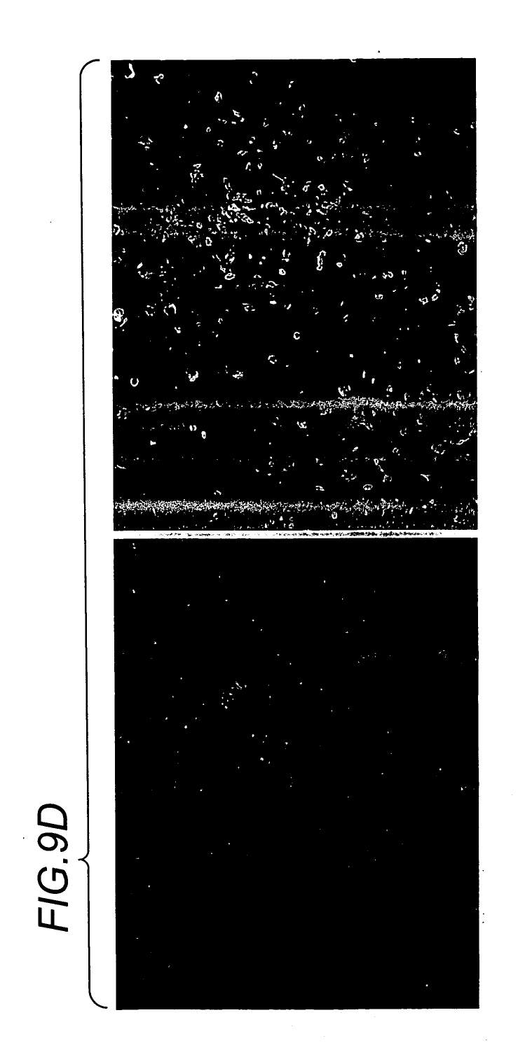

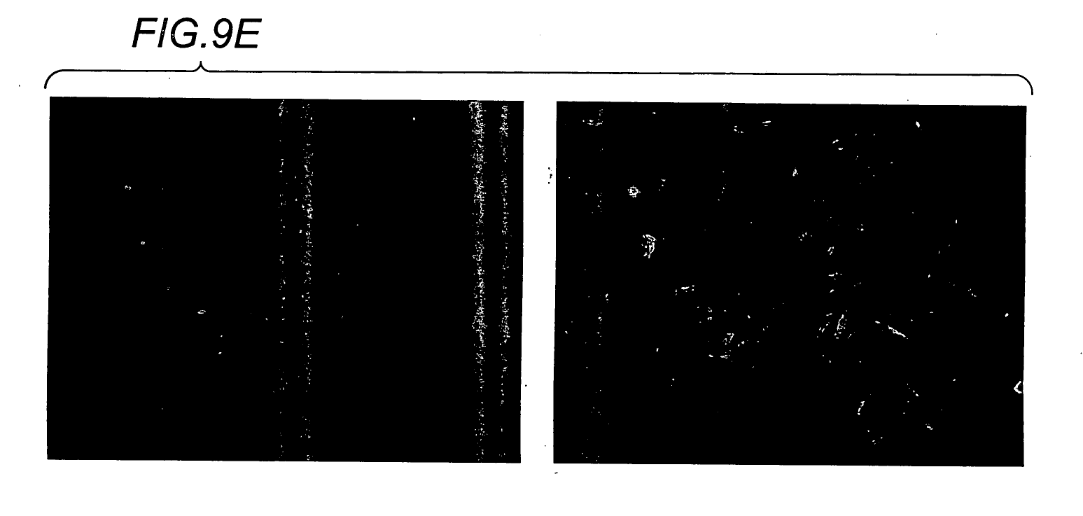

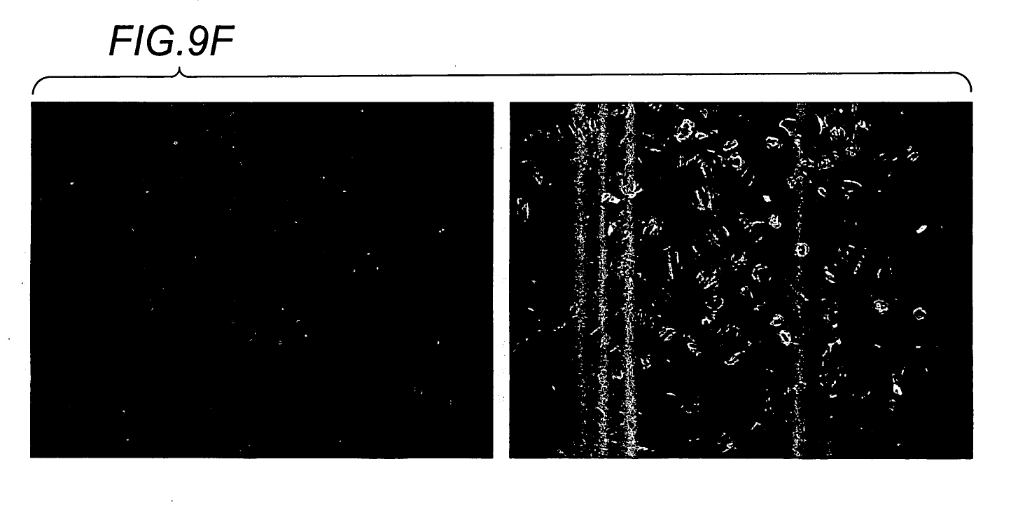

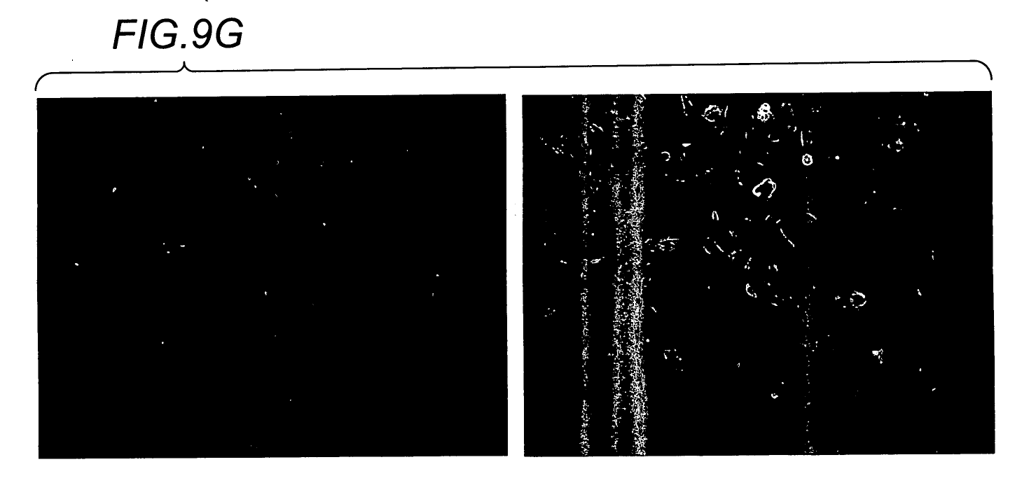

hES cells were cultured on gelatin-coated glass slides using the techniques described in Example 2. The growth media was KO DMEM, 20% heat inactivated fetal bovine serum, L-Glutamine, B-Mercaptoethanol, minimal essential amino acids (Invitrogen, Carlsbad, Calif.). 1 μM retinoic acid (Aldrich) or 300 nM AM580 (a retinoic acid analog, available from Sigma) were added as indicated in FIG. 9. Cells were stained for cytokeratin 7 (red) and DNA (blue) using the techniques described in Examples 3 and 4. Actin (green) was identified by staining with fluorophore-labeled phalloidin (Molecular Probes, Alexa Fluor 488). Cytokeratin 7-positive cells are found even when no growth factor is added, while the addition of a growth factor increases the development of cytokeratin 7-positive cells.

Other embodiments of the invention will be apparent to those skilled in the art from a consideration of the specification or practice of the invention disclosed herein. It is intended that the specification and examples be considered as exemplary only, with the true scope and spirit of the invention being indicated by the following claims.

1. A population of embryonic epithelial cells produced in vitro from embryonic stem cells.

2. The population of claim 1, wherein at least 45% of the cells express cytokeratin.

3. The population of claim 2, wherein at least 55% of the cells express cytokeratin.

4. The population of claim 3, wherein at least 65% of the cells express cytokeratin.

5. The population of claim 4, wherein at least 75% of the cells express cytokeratin.

6. The population of claim 5, wherein at least 85% of the cells express cytokeratin.

7. The population of claim 6, wherein at least 95% of the cells express cytokeratin.

8. The population of claim 1, wherein at least 45% of the cells express cytokeratin 7.

9. A population of cytokeratin-positive cells produced in vitro from embryonic stem cells.

10. The population of claim 9, wherein at least 45% of the cells express cytokeratin.

11. The population of claim 10, wherein at least 55% of the cells express cytokeratin.

12. The population of claim 11, wherein at least 65% of the cells express cytokeratin.

13. The population of claim 12, wherein at least 75% of the cells express cytokeratin.

14. The population of claim 13, wherein at least 85% of the cells express cytokeratin.

15. The population of claim 14, wherein at least 95% of the cells express cytokeratin.

16. The population of claim 9, wherein at least 45% of the cells express cytokeratin 7.

17. A population of cytokeratin-7 positive cells, produced by the step of:
exposing a population of embryonic stem cells to retinoic acid.

18. The population of claim 17, wherein the population is exposed to retinoic acid in the presence of serum.

19. The population of claim 17, wherein the population of embryonic stem cells is seeded on a cell support substrate.

20. The population of claim 18, wherein the cell support substrate comprises polymerized 1,4 butandiol dimethacrylate, diethylene glycol dimethacrylate, phenylene diacrylate 1,3 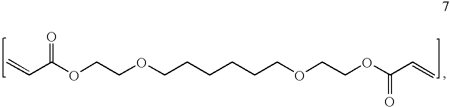
triethylene glycol diacrylate, triethylene glycol dimethacrylate, tripropylene glycol triacrylate 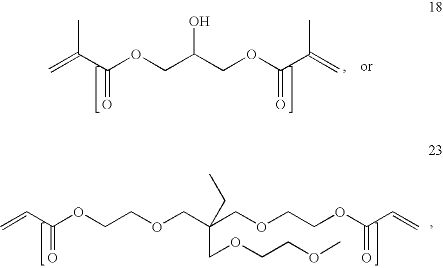
or co-polymers of diethylene glycol methacrylate and 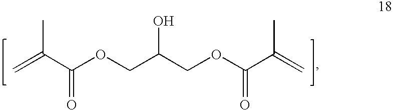
1,4 butanediol dimethacrylate and 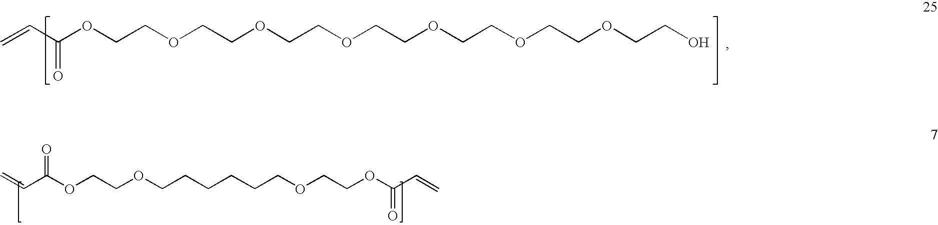
and 1,6, hexanediol diacrylate 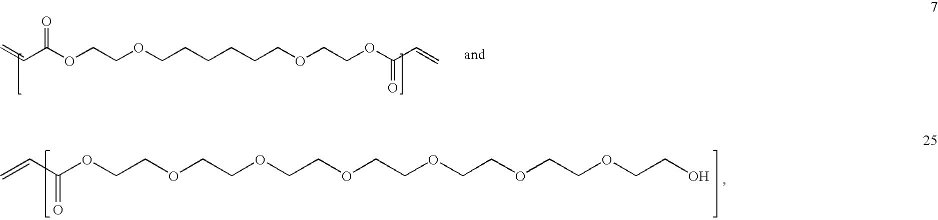
triethylene glycol diacrylate and 1,4 butanediol dimethacrylate, triethylene glycol diacrylate and 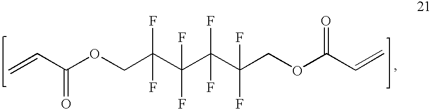
triethylene glycol dimethacrylate and 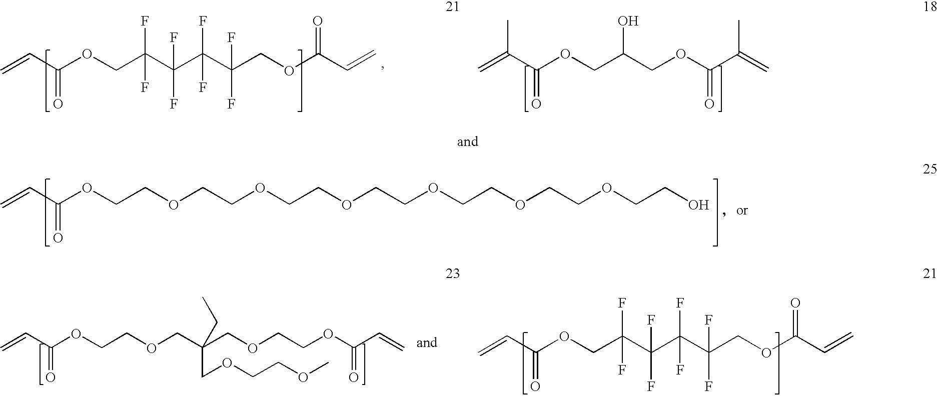
in a ratio of 70/30 by volume.

21. The population of claim 18, wherein the cell support substrate comprises a polymer of one or more monomers having a structure selected from 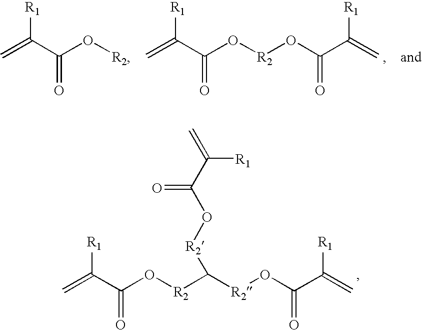
wherein
R₁ is methyl or hydrogen, and
R₂, R₂′, and R₂″ independently include one or more of alkyl, aryl, heterocycle, cycloalkyl, aromatic heterocycle, multicycloalkyl, hydroxyl, ester, ether, halide, carboxylic acid, amino, alkylamino, dialkylamino, trialkylamino, amido, carbamoyl, thioether, thiol, alkoxy, ureido, and branches including one or more of alkyl, aryl, heterocycle, cycloalkyl, aromatic heterocycle, multicycloalkyl, hydroxyl, ester, ether, halide, carboxylic acid, amino, alkylamino, dialkylamino, trialkylamino, amido, carbamoyl, thioether, thiol, alkoxy, and ureido.

22. A composition, comprising:
a cell support substrate; and
human embryonic epithelial cells supported by the cell support substrate.

23. The composition of claim 22, wherein the cell support substrate comprises polymerized 1,4 butanediol dimethacrylate, diethylene glycol dimethacrylate, phenylene diacrylate 1,3, 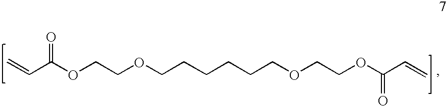
triethylene glycol diacrylate, triethylene glycol dimethacrylate, tripropylene glycol triacrylate, 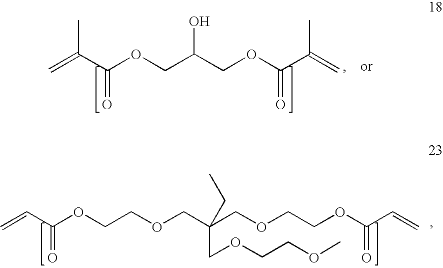
or co-polymers of diethylene glycol methacrylate and 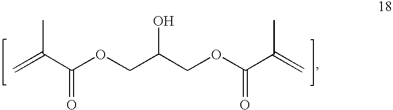
1,4 butanediol dimethacrylate and 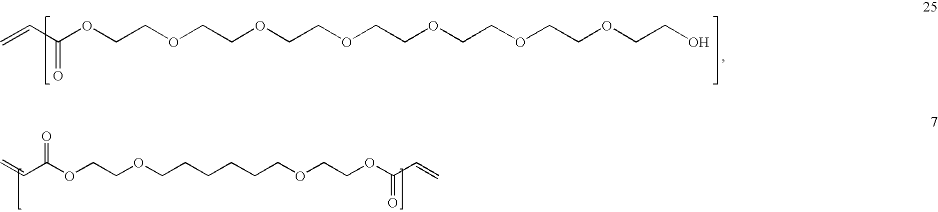
and 1,6, hexanediol diacrylate, 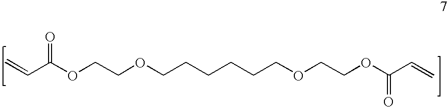
and 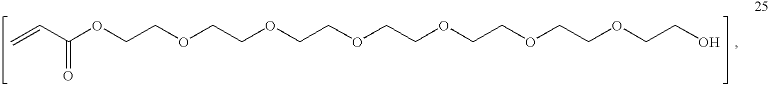
triethylene glycol diacrylate and 1,4 butanediol dimethacrylate, triethylene glycol diacrylate and 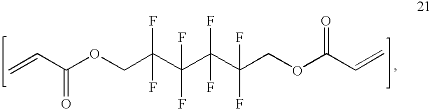
triethylene glycol dimethacrylate and 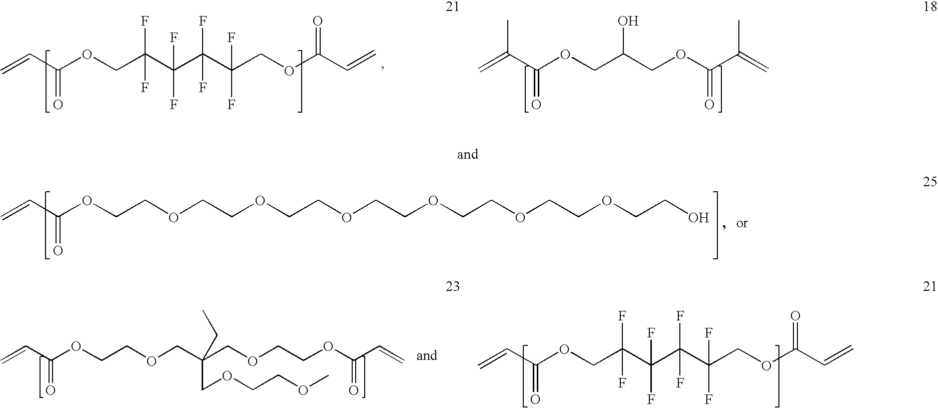
in a ratio of 70/30 by volume.

24. The composition of claim 22, wherein the cell support substrate comprises a polymer of one or more monomers having a structure selected from 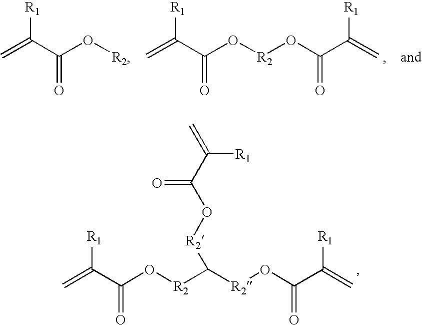
wherein
R₁ is methyl or hydrogen, and
R₂, R₂′, and R₂″ independently include one or more of alkyl, aryl, heterocycle, cycloalkyl, aromatic heterocycle, multicycloalkyl, hydroxyl, ester, ether, halide, carboxylic acid, amino, alkylamino, dialkylamino, trialkylamino, amido, carbamoyl, thioether, thiol, alkoxy, ureido, and branches including one or more of alkyl, aryl heterocycle cycloalkyl, aromatic heterocycle, multicycloalkyl, hydroxyl, ester, ether, ether, halide, carboxylic acid, amino, alkylamino, dialkylamino, trialkylamino, amido, carbamoyl, thioether, thiol, alkoxy, and ureido.

25. The composition of claim 22, further comprising retinoic acid.

26. The composition of claim 22, further comprising serum.

27. The composition of claim 22, wherein at least 45% of the cells express cytokeratin.

28. The composition of claim 27, wherein at least 55% of the cells express cytokeratin.

29. The composition of claim 28, wherein at least 65% of the cells express cytokeratin.

30. The composition of claim 29, wherein at least 75% of the cells express cytokeratin.

31. The composition of claim 30, wherein at least 85% of the cells express cytokeratin.

32. The composition of claim 31, wherein at least 95% of the cells express cytokeratin.

33. The composition of claim 22, wherein at least 45% of the cells express cytokeratin 7.

34. A method of enriching a population of embryonic stem cells with epithelial-like cells, comprising:
providing a population of human embryonic stem cells; and
culturing the stem cells on an acrylate polymer in a culture medium including retinoic acid.

35. The method of claim 34, wherein the culture medium includes serum.

36. The method of claim 34, wherein the acrylate polymer is formed from one or more monomers having a structure selected from 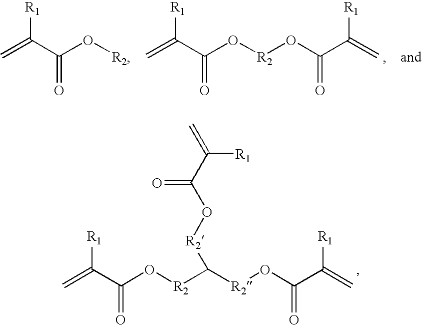
wherein
R₁ is methyl or hydrogen, and
R₂, R₂′, and R₂″ independently include one or more of alkyl, aryl, heterocycle, cycloalkyl, aromatic heterocycle, multicycloalkyl, hydroxyl, ester, ether, halide, carboxylic acid, amino, alkylamino, dialkylamino, trialkylamino, amido, carbamoyl, thioether, thiol, alkoxy, ureido, and branches including one or more of alkyl, aryl, heterocycle, cycloalkyl, aromatic heterocycle, multicycloalkyl, hydroxyl, ester, ether, halide, carboxylic acid, amino, alkylamino, dialkylamino, trialkylamino, amido, carbamoyl, thioether, thiol, alkoxy, and ureido.

37. The method of claim 34, wherein the acrylate polymer is selected from polymerized 1,4 butanediol dimethacrylate, diethylene glycol dimethacrylate, phenylene diacrylate 1,3, 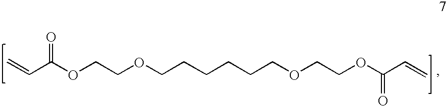
triethylene glycol diacrylate, triethylene glycol dimethacrylate, tripropylene glycol triacrylate 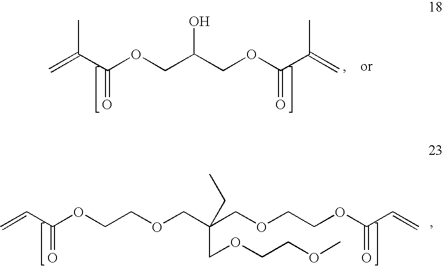
or co-polymers of diethylene glycol methacrylate and 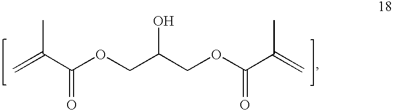
1,4 butanediol dimethacrylate and 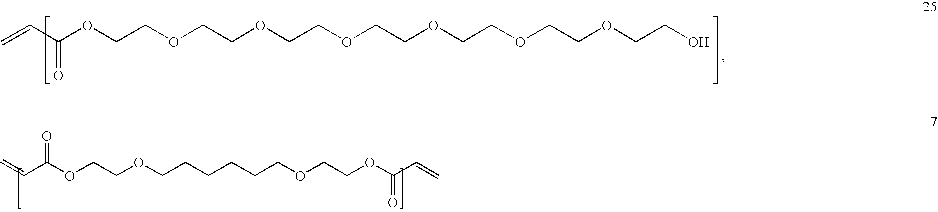
1,6, hexanediol diacrylate, 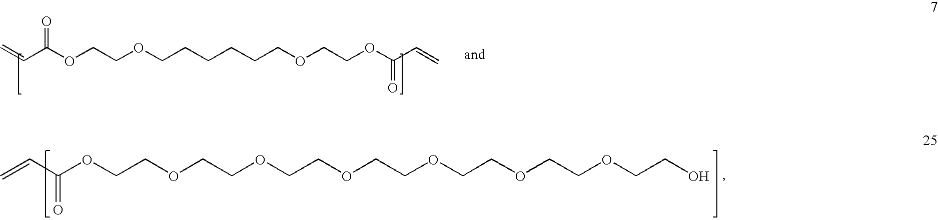
triethylene glycol diacrylate and 1,4 butanediol dimethacrylate, triethylene glycol diacrylate and 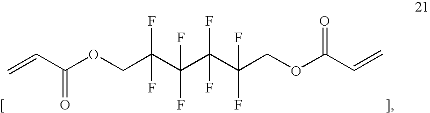
triethylene glycol dimethacrylate and 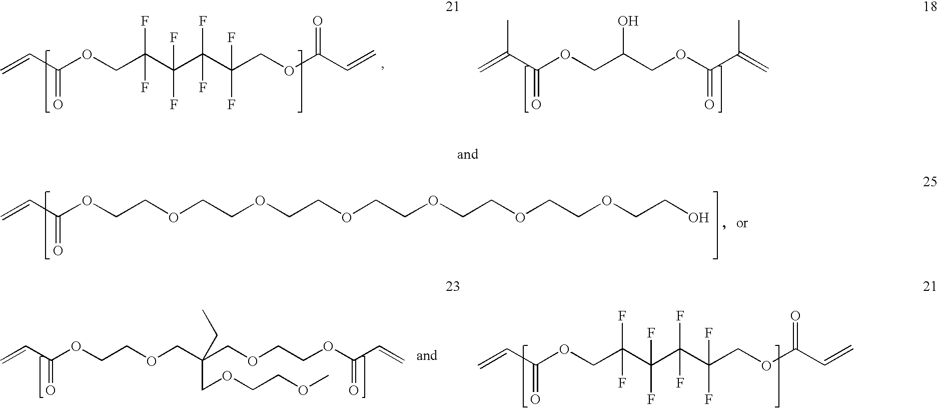
in a ratio of 70/30 by volume.

38. The method of claim 34, wherein providing comprises:
culturing embryonic stem cells under conditions where embryoid bodies are formed; and
dissociating the embryoid bodies.

39. The method of claim 34, wherein at least 45% of the cells express cytokeratin.

40. The method of claim 39, wherein at least 55% of the cells express cytokeratin.

41. The method of claim 40, wherein at least 65% of the cells express cytokeratin.

42. The method of claim 41, wherein at least 75% of the cells express cytokeratin.

43. The method of claim 42, wherein at least 85% of the cells express cytokeratin.

44. The method of claim 43, wherein at least 95% of the cells express cytokeratin.

45. The method of claim 34, wherein at least 45% of the cells express cytokeratin 7.
